(19) United States
(12) Patent Application Publication
(10) Pub. No.: US 2015/0065481 A1
(43) Pub. Date: Mar. 5, 2015

(54) MICROBIOCIDAL PYRAZOLE DERIVATIVES

(71) Applicant: Syngenta Participations AG, Basel (CH)

(72) Inventors: Sarah Sulzer-Mosse, Stein (CH); Clemens Lamberth, Stein (CH); Fredrik Cederbaum, Stein (CH); Dragana Milas, Stein (CH)

(73) Assignee: Syngenta Participations AG, Basel (CH)

(21) Appl. No.: 14/381,028

(22) PCT Filed: Feb. 26, 2013
(86) PCT No.: PCT/EP2013/053815

§ 371 Date: Aug. 26, 2014

(30) Foreign Application Priority Data
Mar. 2, 2012 (EP) 12157850.4

(51) Int. Cl. A01N 43/90; C07D 487/10

(52) U.S. Cl. CPC A01N 43/90; C07D 487/10
USPC 514/210.18; 548/147

(57) ABSTRACT

Compounds of the formula I wherein the substituents areas defined in claim 1, are useful as a pesticides.

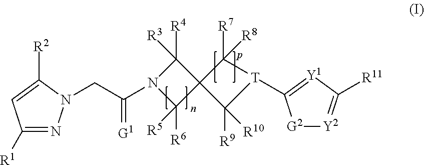

The present invention relates to microbiocidal pyrazole derivatives, e.g. as active ingredients, which have microbiocidal activity, in particular fungicidal activity. The invention also relates to preparation of these pyrazole derivatives, to pyrazole derivatives used as intermediates in the preparation of these pyrazole derivatives, to preparation of these intermediates, to agrochemical compositions which comprise at least one of the pyrazole derivatives, to preparation of these compositions and to use of the pyrazole derivatives or compositions in agriculture or horticulture for controlling or preventing infestation of plants, harvested food crops, seeds or non-living materials by phytopathogenic microorganisms, preferably fungi.

Certain compounds for use as fungicides are described in WO 2007014290, WO 2008013622, WO 2008013925, WO 2008091580, WO 2008091594 and WO 2009055514.

The present invention provides compounds of formula I:

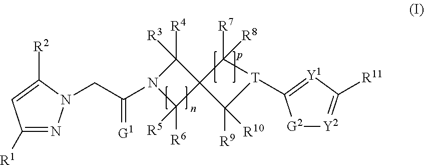

wherein,
G¹ and G² are independently O or S;

### T is CR¹² or N;

Y¹ and Y² are independently CR¹³ or N;
n is 1 or 2;
p is 1 or 2;
R¹ and R² each independently are C₁-C₄alkyl, C₃-C₅cycloalkyl or C₁-C₄haloalkyl;
R³, R⁴, R⁵, R⁶, R⁷, R⁸, R⁹ and R¹⁰ each independently are hydrogen, halogen, cyano, hydroxyl, C₁-C₄alkyl, C₁-C₄haloalkyl and C₁-C₄alkoxy;
R¹¹ is C(═O)—OR¹⁴, C(═O)—NR¹⁴R¹⁵ or a 4- to 15-membered mono-, bi- or tricyclic, saturated, partially unsaturated or aromatic heterocyclic ringsystem optionally substituted by one or more R¹⁶;
R¹² is hydrogen, halogen or hydroxyl;
R¹³ is hydrogen, halogen or cyano;
R¹⁴ is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁶;
R¹⁵ is hydrogen, C₁-C₄alkyl, C₃-C₅cycloalkyl or C₁-C₄alkoxy;
each R¹⁶ independently is halogen, cyano, amino, nitro, hydroxyl, mercapto, C₁-C₈alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkoxy, C₃-C₈cycloalkyl-C₁-C₄alkylthio, C₁-C₈alkoxy, C₃-C₈cycloalkoxy, C₁-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₄alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₃alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁷; and
each R¹⁷ independently is halogen, cyano, C₁-C₄alkyl, C₁-C₄haloalkyl, C₁-C₄alkoxy or C₁-C₄haloalkoxy; or a salt or a N-oxide thereof.

Where substituents are indicated as being optionally substituted, this means that they may or may not carry one or more identical or different substituents, e.g. one to five substituents, e.g. one to three substituents. Normally not more than three such optional substituents are present at the same time. Where a group is indicated as being substituted, e.g. alkyl, unless stated otherwise this includes those groups that are part of other groups, e.g. the alkyl in alkylthio.

The term “halogen” refers to fluorine, chlorine, bromine or iodine, preferably fluorine, chlorine or bromine.

Alkyl substituents may be straight-chained or branched. Alkyl on its own or as part of another substituent is, depending upon the number of carbon atoms mentioned, for example, methyl, ethyl, n-propyl, n-butyl, n-pentyl, n-hexyl and the isomers thereof, for example, iso-propyl, iso-butyl, sec-butyl, tert-butyl, iso-amyl or pivaloyl.

Alkenyl substituents can be in the form of straight or branched chains, and the alkenyl moieties, where appropriate, can be of either the (E)- or (Z)-configuration. Examples are vinyl and allyl. The alkenyl groups are preferably C₂-C₆, more preferably C₂-C₄ and most preferably C₂-C₃ alkenyl groups.

Alkynyl substituents can be in the form of straight or branched chains. Examples are ethynyl and propargyl. The alkynyl groups are preferably C₂-C₆, more preferably C₂-C₄ and most preferably C₂-C₃ alkynyl groups.

Haloalkyl groups may contain one or more identical or different halogen atoms and, for example, may stand for CH₂Cl, CHCl₂, CCl₃, CH₂F, CHF₂, CF₃, CF₃CH₂, CH₃CF₂, CF₃CF₂ or CCl₃CCl₂.

Haloalkenyl groups are alkenyl groups, respectively, which are substituted with one or more of the same or different halogen atoms and are, for example, 2,2-difluorovinyl or 1,2-dichloro-2-fluoro-vinyl.

Haloalkynyl groups are alkynyl groups, respectively, which are substituted with one or more of the same or different halogen atoms and are, for example, 1-chloro-prop-2-ynyl.

Alkoxy means a radical —OR, where R is alkyl, e.g. as defined above. Alkoxy groups include, but are not limited to, methoxy, ethoxy, 1-methylethoxy, propoxy, butoxy, 1-methylpropoxy and 2-methylpropoxy.

Cyano means a —CN group.

Amino means an NH₂ group.

Hydroxyl or hydroxy stands for a OH group.

Aryl means a ring system which may be mono-, bi- or tricyclic. Examples of such rings include phenyl, naphthalenyl, anthracenyl, indenyl or phenanthrenyl. A preferred aryl group is phenyl.

Heteroaryl stands for aromatic ring systems comprising mono-, bi- or tricyclic systems wherein at least one oxygen, nitrogen or sulfur atom is present as a ring member. Monocyclic and bicyclic aromatic ring systems are preferred, monocyclic ring systems are more preferred. For example, monocyclic heteroaryl may be a 5- to 7-membered aromatic ring containing one to three heteroatoms selected from oxygen, nitrogen and sulfur, more preferably selected from nitrogen and sulfur. Bicyclic heteroaryl may be a 9- to 11-membered bicyclic ring containing one to five heteroatoms, preferably one to three heteroatoms, selected from oxygen, nitrogen and sulfur. Examples are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, imiazothiazoyl, quinolinyl, quinoxalinyl, isoquinolinyl, phthalazinyl, quinoxalinyl, quinazolinyl, cinnolinyl and naphthyridinyl, preferably pyridyl, pyrazinyl, pyridazinyl, pyrimidinyl, pyrrolyl, pyrazolyl, imidazolyl, triazolyl, furanyl, thienyl thiazolyl or thiadiazolyl. Heteroaryl rings do not contain adjacent oxygen ring atoms, adjacent sulfur ring atoms or adjacent oxygen and sulfur ring atoms. A link to a heteroaryl group can be via a carbon atom or via a nitrogen atom.

Heterocyclyl is defined to include heteroaryl and in addition their saturated or partially unsaturated analogues. The different rings of bi- or tricyclic heterocyclic ring systems may be linked via one atom belonging to two different rings (spiro), via two adjacent ring atoms belonging to two different rings (annelated) or via two different, not adjacent ring atoms belonging to two different rings (bridged).

The presence of one or more possible asymmetric carbon atoms in a compound of formula I means that the compounds may occur in optically isomeric forms, i.e. enantiomeric or diastereomeric forms. Also atropisomers may occur as a result of restricted rotation about a single bond. Formula I is intended to include all those possible isomeric forms and mixtures thereof. The present invention includes all those possible isomeric forms and mixtures thereof for a compound of formula I. Likewise, formula I is intended to include all possible tautomers. The present invention includes all possible tautomeric forms for a compound of formula I.

In each case, the compounds of formula I according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The following list provides definitions, including preferred definitions, for substituents G¹, G², T, Y¹, Y², n, p, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ with reference to compounds of formula I and other compounds of the invention carrying the same substituents. For any one of these substituents, any of the definitions given below may be combined with any definition of any other substituent given below or elsewhere in this document.

G¹ and G² are independently O or S.

G¹ is preferably O.

G² is preferably S.

T is CR¹² or N, preferably CH or N, more preferably CH.

Y¹ and Y² are independently CR¹³ or N.

Y¹ is preferably CH or N, more preferably N.

Y² is preferably CH or N; more preferably CH.

n is 1 or 2, preferably 1.

p is 1 or 2, preferably 1.

Preferably, p is 1 and n is 1.

R¹ and R² each independently are C₁-C₄alkyl, C₃-C₅cycloalkyl or C₁-C₄haloalkyl. Preferably R¹ and R² are each independently methyl or halomethyl, more preferably methyl, difluoromethyl or trifluoromethyl.

Preferably R¹ is difluoromethyl or trifluoromethyl. Preferably R² is methyl or difluoromethyl. In one group of compounds R¹ is trifluoromethyl and R² is methyl. In another group of compounds R¹ and R² are both difluoromethyl.

R³, R⁴, R⁵, R⁶, R⁷, R⁸, R⁹ and R¹⁰ each independently are hydrogen, halogen, cyano, hydroxyl, C₁-C₄alkyl, C₁-C₄haloalkyl and C₁-C₄alkoxy.

Preferably R³, R⁴, R⁵, R⁶, R⁷, R⁸, R⁹ and R¹⁰ each independently are hydrogen, halogen, hydroxyl and C₁-C₄alkyl;

More preferably R³, R⁴, R⁵, R⁶, R⁷, R⁸, R⁹ and R¹⁰ are hydrogen.

R¹¹ is C(═O)—OR¹⁴, C(═O)—NR¹⁴R¹⁵ or a 4- to 15-membered mono-, bi- or tricyclic, saturated, partially unsaturated or aromatic heterocyclic ringsystem optionally substituted by one or more R¹⁶. Preferably R¹¹ is C(═O)—NR¹⁴R¹⁵ or a 5-membered heterocyclic ringsystem optionally substituted by one or more R¹⁶. More preferably R¹¹ is C(═O)—NR¹⁴R¹⁵ or a isoxazoline optionally substituted by one or more R¹⁶.

R¹² is hydrogen, halogen or hydroxyl; preferably hydrogen, fluoro or hydroxyl, more preferably hydrogen.

R¹³ is hydrogen, halogen or cyano; preferably hydrogen or cyano, more preferably hydrogen.

R¹⁴ is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁶. Preferably R¹⁴ is arylalkyl or a 9- to 10-membered bicyclic carbocyclic ringsystem, wherein the arylalkyl and 9- to 10-membered bicyclic carbocyclic ringsystem are optionally substituted by one or more R¹⁶. More preferably R¹⁴ is benzyl or a 10-membered bicyclic carbocyclic ringsystem, wherein the benzyl and 10-membered bicyclic carbocyclic ringsystem are optionally substituted by one or more R¹⁶.

R¹⁵ is hydrogen, C₁-C₄alkyl, C₃-C₅cycloalkyl or C₁-C₄alkoxy. Preferably R¹⁵ is hydrogen or C₁-C₄alkyl. More preferably R¹⁵ is hydrogen or methyl.

Each R¹⁶ is independently, halogen, cyano, amino, nitro, hydroxyl, mercapto, C₁-C₈ alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkyloxy, C₃-C₈cycloalkyl-C₁-C₄alkylthio, C₁-C₈alkoxy, C₃-C₈cycloalkyloxy, C₂-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₄alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₃alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein the aryl and heterocyclyl are optionally substituted by one or more R¹⁷.

Preferably each R¹⁶ independently is halogen, cyano, amino, nitro, hydroxyl, mercapto, C₁-C₈ alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkylthio, C₁-C₈alkoxy, C₃-C₈cycloalkoxy, C₂-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, phenyl, phenyloxy, phenylthio, phenylsulfonyl, phenylsulfinyl, phenyl-C₁-C₄alkyl, phenyl-C₁-C₄alkyloxy, phenyl-C₁-C₄alkylthio, heterocyclyl, heterocycyl-C₁-C₄alkyl, heterocycyl-C₁-C₄alkyloxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁷; and wherein heterocyclyl is independently selected from pyridyl, pyrazinyl, pyridazinyl, pyrimidinyl, pyrrolyl, pyrazolyl, imidazolyl, triazolyl, furanyl, thienyl, thiazolyl, thiadiazolyl, pyrrolidinyl, piperazinyl, piperidinyl, morpholinyl, and tetrahydropyranyl.

More preferably each R¹⁶ independently is halogen, cyano, amino, mercapto, C₁-C₈alkyl, C₃-C₈cycloalkyl, C₃-C₈cycloalkyl-C₁-C₄alkyloxy, C₃-C₈cycloalkyl-C₁-C₄alkylthio, C₁-C₈alkoxy, C₁-C₈alkylthio, phenyl, phenyloxy, phenylthio, phenyl-C₁-C₄alkoxy, phenyl-C₁-C₄alkylthio, heterocyclyl, heterocyclyl-C₁-C₄alkoxy, heterocyclyl-C₁-C₄alkylthio, NH(C₁-C₈alkyl), N(C₁-C₈alkyl)₂, and wherein heterocyclyl is independently selected from pyridyl, pyrazinyl, pyridazinyl, pyrimidinyl, pyrrolyl, pyrazolyl, imidazolyl, triazolyl, furanyl, thienyl, thiazolyl, thiadiazolyl, pyrrolidinyl, piperazinyl, piperidinyl, morpholinyl, and tetrahydroyranyl, and wherein alkyl, cycloalkyl and alkoxy are optionally substituted by halogen, and wherein aryl and heterocyclyl moieties are optionally substituted by one or more R¹⁷.

Even more preferably each R¹⁶ independently is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁷.

Each R¹⁷ is independently halogen, cyano, C₁-C₄alkyl, C₁-C₄haloalkyl, C₁-C₄alkoxy or C₁-C₄haloalkoxy, preferably each R¹⁷ independently is halogen, cyano, methyl, halomethyl, methoxy or halomethoxy, more preferably each R¹⁷ independently is halogen, methyl or halomethyl.

The compound of the invention may be a compound of formula I-a

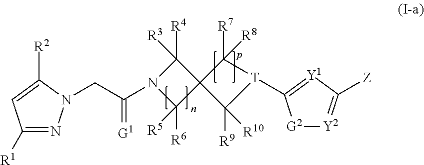

wherein Z is selected from Z-1 to Z-12

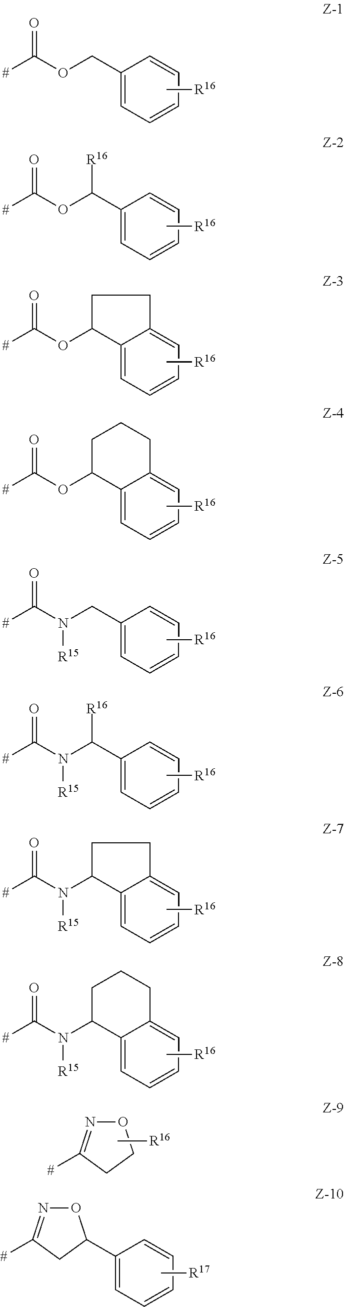

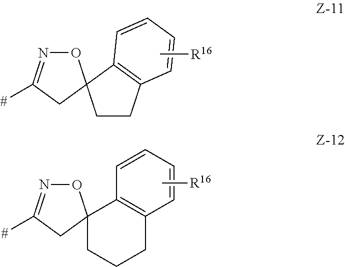

and G¹, G², T, Y¹, Y², n, p, R¹, R², R³, R⁴, R⁵, R⁶, R⁷, R⁸, R⁹, R¹⁰, R¹⁵, R¹⁶ and R¹⁷ are as defined for formula I. The preferred substituent definitions are the same as for compounds of formula I. Preferably Z is selected from Z-1, Z-4, Z-8, Z-10 and Z-12.

Preferably, in compounds according to formula I-a, R¹⁶ and R¹⁷ are independently C₁-C₄alkyl, C₁-C₄haloalkyl or halogen.

In one group of compounds of the invention G¹ and G² are independently O or S; T is CR¹² or N; Y¹ is N; Y² is CR¹³ or N; n is 1; p is 1; R¹ and R² each independently are C₁-C₄alkyl, C₃-C₅cycloalkyl or C₁-C₄haloalkyl; R³, R⁴, R⁵, R⁶, R⁷, R⁸, R⁹ and R¹⁰ each independently are hydrogen, halogen, hydroxyl and C₁-C₄alkyl; R¹¹ is C(═O)—OR¹⁴, C(═O)—NR¹⁴R¹⁵ or a 4- to 15-membered mono-, bi- or tricyclic, saturated, partially unsaturated or aromatic heterocyclic ringsystem optionally substituted by one or more R¹⁶; R¹² is hydrogen, halogen or hydroxyl; R¹³ is hydrogen, halogen or cyano; R¹⁴ is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁶; R¹⁵ is hydrogen, C₁-C₄alkyl, C₃-C₅cycloalkyl or C₁-C₄alkoxy; each R¹⁶ independently is halogen, cyano, amino, nitro, hydroxyl, mercapto, C₁-C₈alkyl, C₂-C₃alkenyl, C₂-C₈alkynyl, C₃-C₈cycloalkyl, C₃-C₈cycloalkyl-C₁-C₄alkyl, C₃-C₈cycloalkyl-C₁-C₄alkoxy, C₃-C₈cycloalkyl-C₁-C₄alkylthio, C₁-C₈alkoxy, C₃-C₈cycloalkoxy, C₁-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₄alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₃cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁷; and

each R¹⁷ independently is halogen, cyano, C₁-C₄alkyl, C₁-C₄haloalkyl, C₁-C₄alkoxy or C₁-C₄haloalkoxy.

In another group of compounds of the invention G¹ is O; G² is S; T is CH or N; Y¹ is N; Y² is CH; n is 1; p is 1; R¹ is difluoromethyl or trifluoromethyl; R² is methyl or difluoromethyl; R³, R⁴, R⁵, R⁶, R⁷, R⁸, R⁹ and R¹⁹ each independently are hydrogen, halogen, cyano, hydroxyl, C₁-C₄alkyl, C₁-C₄haloalkyl and C₁-C₄alkoxy; R¹¹ is C(═O)—NR¹⁴R¹⁵ or a 5-membered heterocyclic ringsystem optionally substituted by one or more R¹⁶; R¹⁴ is arylalkyl or a 9- to 10-membered bicyclic carbocyclic ringsystem, wherein the arylalkyl and 9- to 10-membered bicyclic carbocyclic ringsystem are optionally substituted by one or more R¹⁶; R¹⁵ is hydrogen or methyl; each R¹⁶ is halogen, cyano, amino, mercapto, C₁-C₄alkyl, C₃-C₆cycloalkyl, C₃-C₈cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁷ and R¹⁷ is halogen, methyl or halomethyl.

In another group of compounds of the invention G¹ is O; G² is S; T is CH or N; Y¹ is N; Y² is CH; n is 1; p is 1; R¹ is difluoromethyl or trifluoromethyl; R² is methyl or difluoromethyl; R³, R⁴, R⁵, R⁶, R⁷, R⁸, R⁹ and R¹⁰ each independently are hydrogen, halogen, hydroxyl and C₁-C₄alkyl; R¹¹ is C(═O)—NR¹⁴R¹⁵ or a 5-membered heterocyclic ringsystem optionally substituted by one or more R¹⁶; R¹⁴ is arylalkyl or a 9- to 10-membered bicyclic carbocyclic ringsystem, wherein the arylalkyl and 9- to 10-membered bicyclic carbocyclic ringsystem are optionally substituted by one or more R¹⁶; R¹⁵ is hydrogen or methyl; each R¹⁶ is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁷ and R¹⁷ is halogen, methyl or halomethyl.

In another group of compounds the compound of the invention is a compound of formula I.a, wherein G¹ is O; G² is S; T is CH or N; Y¹ is N; Y² is CH; Z is selected from Z-1 to Z-12 (above); preferably Z-1, Z-4, Z-8, Z-10 and Z-12; n is 1; p is 1; R¹ is difluoromethyl or trifluoromethyl; R² is methyl or difluoromethyl; and R³, R⁴, R⁵, R⁶, R⁷, R⁸, R⁹ and R¹⁰ each independently are hydrogen, halogen, cyano, hydroxyl, C₁-C₄alkyl, C₁-C₄haloalkyl and C₁-C₄alkoxy.

In another group of compounds the compound of the invention is a compound of formula I.a, wherein G¹ is O; G² is S; T is CH or N; Y¹ is N; Y² is CH; Z is selected from Z-1 to Z-12 (above); preferably Z-1, Z-4, Z-8, Z-10 and Z-12; n is 1; p is 1; R¹ is difluoromethyl or trifluoromethyl; R² is methyl or difluoromethyl; and R³, R⁴, R⁵, R⁶, R⁷, R⁸, R⁹ and R¹⁰ each independently are hydrogen, halogen, hydroxyl and C₁-C₄alkyl.

For the avoidance of doubt, when n is 1 and p is 1 compounds of formula I have the formula according to formula I-b:

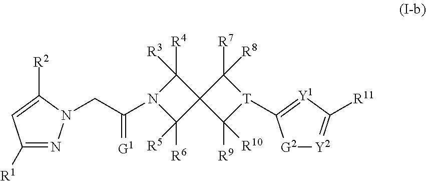

in which R¹, R², R³, R⁴, R⁵, R⁶, R⁷, R⁸, R⁹, R¹⁰, R¹¹, G¹, G², T, Y¹ and Y² have the definitions as described for formula I.

When n is 2 and p is 1, compounds of formula I have the formula according to formula I-c:

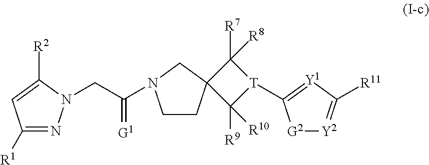

in which R¹, R², R³, R⁴, R⁵, R⁶, R⁷, R⁸, R⁹, R¹⁰, R¹¹, G¹, G², T, Y¹ and Y² have the definitions as described for formula I.

When n is 1 and p is 2, compounds of formula I have the formula according to formula I-d:

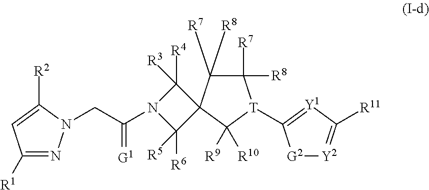

in which R¹, R², R³, R⁴, R⁵, R⁶, R⁷, R⁸, R⁹, R¹⁰, R¹¹, G¹, G², T, Y¹ and Y² have the definitions as described for formula I.

The invention also relates to compounds of formula I-b, formula I-c and formula I-d as shown above.

The invention also relates to compounds of formula I-e:

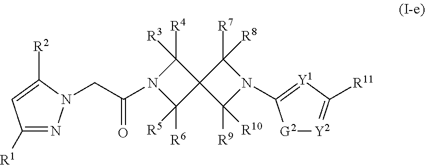

in which R¹, R², R³, R⁴, R⁵, R⁶, R⁷, R⁸, R⁹, R¹⁰, R¹¹, G², Y¹ and Y² have the definitions as described for formula I. Preferred definitions of R¹, R², R³, R⁴, R⁵, R⁶, R⁷, R⁸, R⁹, R¹⁰, R¹¹, G², Y¹ and Y² are as defined for formula I.

The invention also relates to compounds of formula I-f:

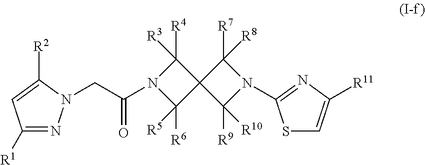

in which R¹, R², R³, R⁴, R⁵, R⁶, R⁷, R⁸, R⁹, R¹⁰ and R¹¹ have the definitions as described for formula I. Preferred definitions of R¹, R², R³, R⁴, R⁵, R⁶, R⁷, R⁸, R⁹, R¹⁰ and R¹¹ are as defined for formula I.

The invention also relates to compounds of formula I-g:

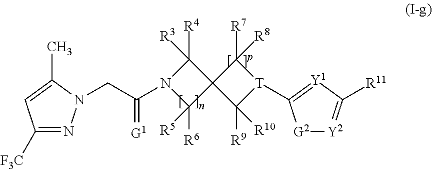

wherein R³, R⁴, R⁵, R⁶, R⁷, R⁸, R⁹, R¹⁰, R¹¹, G¹, G², T, Y¹, Y², n and p have the definition as described for formula I. Preferred definitions of R³, R⁴, R⁵, R⁶, R⁷, R⁸, R⁹, R¹⁰, R¹¹, G¹, G², T, Y¹, Y², n and p are as defined for formula I.

The invention also relates to compounds of formula I-h:

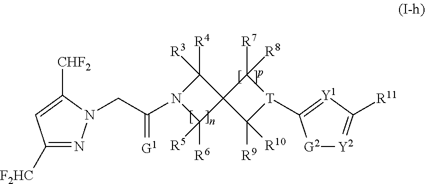

in which R³, R⁴, R⁵, R⁶, R⁷, R⁸, R⁹, R¹⁰, R¹¹, G¹, G², T, Y¹, Y², n and p have the definitions as described for formula I. Preferred definitions of R³, R⁴, R⁵, R⁶, R⁷, R⁸, R⁹, R¹⁰, R¹¹, G¹, G², T, Y¹, Y², n and p are as defined for formula I.

The invention includes compounds of formula II:

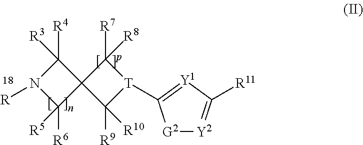

wherein R³, R⁴, R⁵, R⁶, R⁷, R⁸, R⁹, R¹⁰, R¹¹, G², T, Y¹, Y², n and p are as defined for a compound of formula I and R¹⁸ is hydrogen, a protecting group, such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl. These compounds, including salts and N-oxides thereof, are useful as intermediates in the synthesis of compounds of formula I. Preferred definitions of R³, R⁴, R⁵, R⁶, R⁷, R⁸, R⁹, R¹⁰, R¹¹, G², T, Y¹, Y², n and p are as defined for formula I.

The invention also includes compounds of formula III

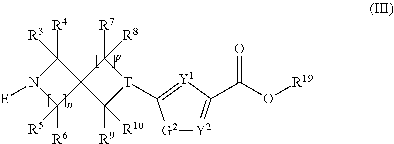

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M,

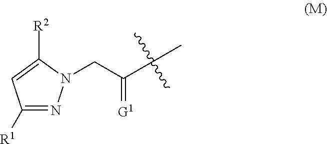

R¹, R², R³, R⁴, R⁵, R⁶, R⁷, R⁸, R⁹, R¹⁰, G¹, G², T, Y¹, Y², n and p are as defined for a compound of formula I and R¹⁹ is C₁-C₆alkyl. These compounds, including salts and N-oxides thereof, are useful as intermediates in the synthesis of compounds of formula I. Preferred definitions of R¹, R², R³, R⁴, R⁵, R⁶, R⁷, R⁸, R⁹, R¹⁰, G¹, G², T, Y¹, Y², n and p a are as defined for formula I.

The invention also includes compounds of formula IV

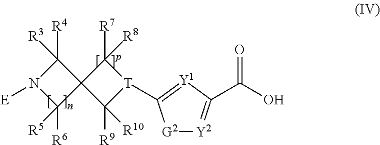

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M

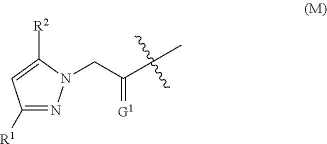

and R¹, R², R³, R⁴, R⁵, R⁶, R⁷, R⁸, R⁹, R¹⁰, G¹, G², T, Y¹, Y², n and p are as defined for a compound of formula I. These compounds, including salts and N-oxides thereof, are useful as intermediates in the synthesis of compounds of formula I. Preferred definitions of R¹, R², R³, R⁴, R⁵, R⁶, R⁷, R⁸, R⁹, R¹⁰, G¹, G², T, Y¹, Y², n and p are as defined for formula I.

The invention also includes compounds of formula V

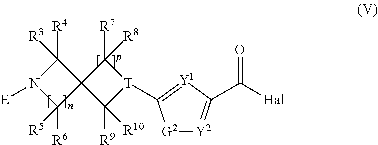

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M,

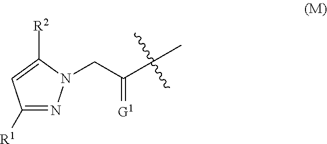

R¹, R², R³, R⁴, R⁵, R⁶, R⁷, R⁸, R⁹, R¹⁰, G¹, G², T, Y¹, Y², n and p are as defined for a compound of formula I and Hal is halogen, preferably chloro. These compounds, including salts and N-oxides thereof, are useful as intermediates in the synthesis of compounds of formula I. Preferred definitions of R¹, R², R³, R⁴, R⁵, R⁶, R⁷, R⁸, R⁹, R¹⁰, G¹, G², T, Y¹, Y², n and p are as defined for formula I.

The invention also includes compounds of formula VI

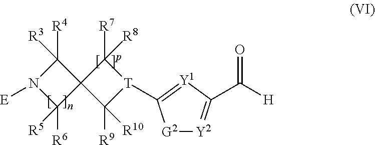

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M

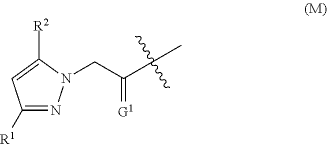

and R¹, R², R³, R⁴, R⁵, R⁶, R⁷, R⁸, R⁹, R¹⁰, G¹, G², T, Y¹, Y², n and p are as defined for a compound of formula I. These compounds, including salts and N-oxides thereof, are useful as intermediates in the synthesis of compounds of formula I. Preferred definitions of R¹, R², R³, R⁴, R⁵, R⁶, R⁷, R⁸, R⁹, R¹⁰, G¹, G², T, Y¹, Y², n and p are as defined for formula I.

Preferred individual compounds of formula I are:
2-{2-[2-(5-methyl-3-trifluoromethyl-pyrazol-1-yl)-acetyl]-2-aza-spiro[3.3]hept-6-yl}-thiazole-4-carboxylic acid 2-chloro-6-fluoro-benzyl ester;
2-{2-[2-(3,5-bis-difluoromethyl-pyrazol-1-yl)-acetyl]-2-aza-spiro[3.3]hept-6-yl}-thiazole-4-carboxylic acid 2-chloro-6-fluoro-benzyl ester;
2-{6-[2-(5-methyl-3-trifluoromethyl-pyrazol-1-yl)-acetyl]-2,6-diaza-spiro[3.3]hept-2-yl}-thiazole-4-carboxylic acid 2-chloro-6-fluoro-benzyl ester;
2-{6-[2-(3,5-bis-difluoromethyl-pyrazol-1-yl)-acetyl]-2,6-diaza-spiro[3.3]hept-2-yl}-thiazole-4-carboxylic acid 2-chloro-6-fluoro-benzyl ester;
2-{2-[2-(5-methyl-3-trifluoromethyl-pyrazol-1-yl)-acetyl]-2-aza-spiro[3.3]hept-6-yl}-thiazole-4-carboxylic acid methyl-(1,2,3,4-tetrahydro-naphthalen-1-yl)-amide;
2-{2-[2-(3,5-bis-difluoromethyl-pyrazol-1-yl)-acetyl]-2-aza-spiro[3.3]hept-6-yl}-thiazole-4-carboxylic acid methyl-(1,2,3,4-tetrahydro-naphthalen-1-yl)-amide;
2-{6-[2-(5-methyl-3-trifluoromethyl-pyrazol-1-yl)-acetyl]-2,6-diaza-spiro[3.3]hept-2-yl}-thiazole-4-carboxylic acid methyl-(1,2,3,4-tetrahydro-naphthalen-1-yl)-amide;
2-{6-[2-(3,5-bis-difluoromethyl-pyrazol-1-yl)-acetyl]-2,6-diaza-spiro[3.3]hept-2-yl}-thiazole-4-carboxylic acid methyl-(1,2,3,4-tetrahydro-naphthalen-1-yl)-amide;
1-(6-{4-[5-(2,6-difluoro-phenyl)-4,5-dihydro-isoxazol-3-yl]-thiazol-2-yl}-2-aza-spiro[3.3]hept-2-yl)-2-(5-methyl-3-trifluoromethyl-pyrazol-1-yl)-ethanone;
2-(3,5-bis-difluoromethyl-pyrazol-1-yl)-1-(6-{4-[5-(2,6-difluoro-phenyl)-4,5-dihydro-isoxazol-3-yl]-thiazol-2-yl}-2-aza-spiro[3.3]hept-2-yl)-ethanone;
1-(6-{4-[5-(2,6-difluoro-phenyl)-4,5-dihydro-isoxazol-3-yl]-thiazol-2-yl}-2,6-diaza-spiro[3.3]hept-2-yl)-2-(5-methyl-3-trifluoromethyl-pyrazol-1-yl)-ethanone; and
2-(3,5-bis-difluoromethyl-pyrazol-1-yl)-1-(6-{4-[5-(2,6-difluoro-phenyl)-4,5-dihydro-isoxazol-3-yl]-thiazol-2-yl}-2,6-diaza-spiro[3.3]hept-2-yl)-ethanone.

Compounds of the present invention can be made as shown in the following schemes. Throughout this description, the group M, wherein R¹, R² and G¹ are as defined for formula I, stands for:

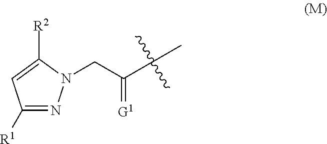

The compounds of formula VIII, wherein R³, R⁴, R₅, R⁶, R⁷, R⁸, R⁹, R¹⁰, R¹⁴, G², T, Y¹, Y², n and p are as defined for formula I and E is hydrogen, a protecting group such as acetyl, benzyl or tert-butoxycarbonyl or a group M, can be obtained by transformation of a compound of formula V, wherein R³, R⁴, R⁵, R⁶, R⁷, R⁸, R⁹, R¹⁰, G², T, Y¹, Y², n and p are as defined for formula I, Hal is halogen, preferably chloro, and E is hydrogen, a protecting group such as acetyl, benzyl or tert-butoxycarbonyl or a group M, with a compound of formula VII, wherein R¹⁴ is as defined for formula I and a base, such as pyridine, triethylamine, ethyl diisopropylamine. This is shown in Scheme 1.

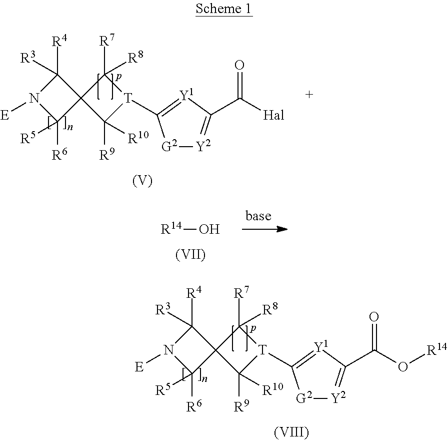

The compounds of formula V, wherein R³, R⁴, R⁵, R⁶, R⁷, R⁸, R⁹, R¹⁰, G², T, Y¹, Y², n and p are as defined for formula I, Hal is halogen, preferably chloro, and E is hydrogen, a protecting group such as acetyl, benzyl or tert-butoxycarbonyl or a group M, can be obtained by transformation of a compound of formula IV, wherein R³, R⁴, R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with a thionyl halide, such as thionyl chloride, or a phosphoryl halide, such as phosphoryl chloride. This is shown in Scheme 2.

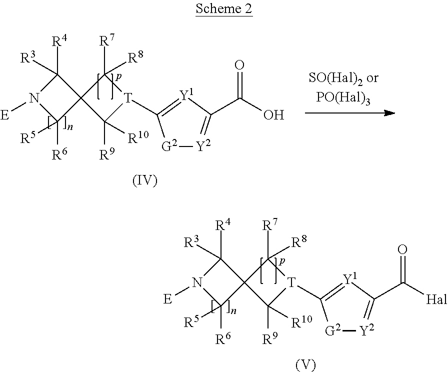

The compounds of formula IV, wherein R³, R⁴, R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, can be obtained by transformation of a compound of formula III, wherein R³, R⁴, R⁵, R⁶, R⁷, R⁸, R⁹, R¹⁰, G², T, Y¹, Y², n and p are as defined for formula I, R¹⁹ is C₁-C₆alkyl and E is hydrogen, a protecting group such as acetyl, benzyl or tert-butoxycarbonyl or a group M, with a base, such as lithium hydroxide, sodium hydroxide, sodium carbonate, potassium hydroxide or potassium carbonate. This is shown in Scheme 3.

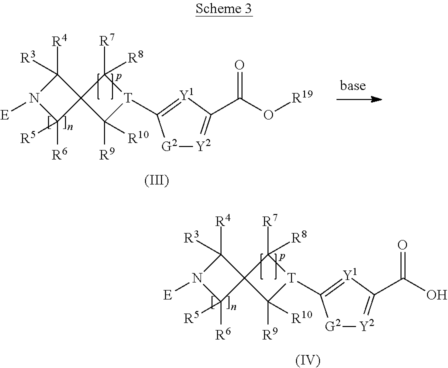

Alternatively, the compounds of formula VIII, wherein R³, R⁴, R⁵, R⁶, R⁷, R⁸, R⁹, R¹⁰, R¹⁴, G², T, Y¹, Y², n and p are as defined for formula I and E is hydrogen, a protecting group such as acetyl, benzyl or tert-butoxycarbonyl or a group M, can be obtained by transformation of a compound of formula IV, wherein R³, R⁴, R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with a compound of formula VII, wherein R¹⁴ is as defined for formula I and an acid, such as hydrochloric acid or sulphuric acid. This is shown in Scheme 4.

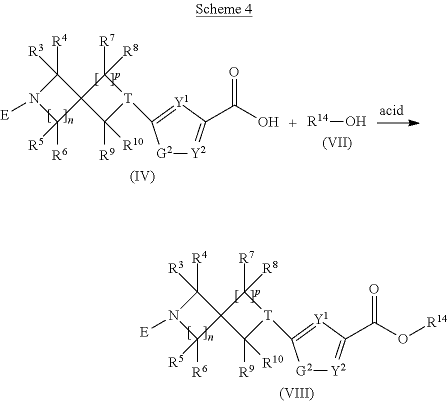

The compounds of formula X, wherein R³, R⁴, R⁵, R⁶, R⁷, R⁸, R⁹, R¹⁰, R¹⁴, R¹⁵, G², T, Y¹, Y², n and p are as defined for formula I and E is hydrogen, a protecting group such as acetyl, benzyl or tert-butoxycarbonyl or a group M, can be obtained by transformation of a compound of formula V, wherein R³, R⁴, R⁵, R⁶, R⁷, R⁸, R⁹, R¹⁰, G², T, Y¹, Y², n and p are as defined for formula I, Hal is halogen, preferably chloro, and E is hydrogen, a protecting group such as acetyl, benzyl or tert-butoxycarbonyl or a group M, with a compound of formula IX, wherein R¹⁴ and R¹⁵ are as defined for formula I and a base, such as pyridine, triethylamine, ethyl diisopropylamine. This is shown in Scheme 5.

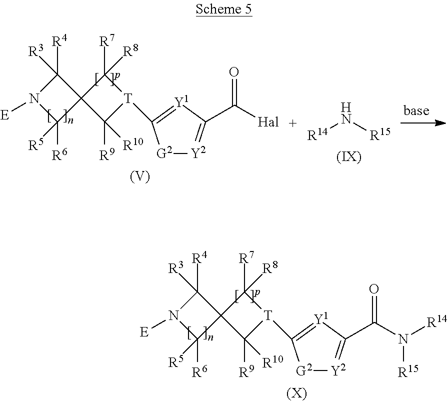

Alternatively, the compounds of formula X, wherein R³, R⁴, R⁵, R⁶, R⁷, R⁸, R⁹, R¹⁰, R¹⁴, R¹⁵, G², T, Y¹, Y², n and p are as defined for formula I and E is hydrogen, a protecting group such as acetyl, benzyl or tert-butoxycarbonyl or a group M, can be obtained by transformation of a compound of formula IV, wherein R³, R⁴, R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with a compound of formula IX, wherein R¹⁴ and R¹⁵ are as defined for formula I, an activating reagent, such as BOP, PyBOP, EDCl, CDI or HATU, and a base, such as pyridine, triethylamine, ethyl diisopropylamine. This is shown in Scheme 6.

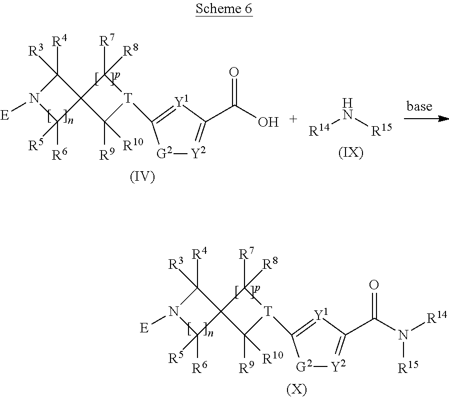

The compounds of formula XII, wherein R³, R⁴, R⁵, R⁶, R⁷, R⁸, R⁹, R¹⁰, R¹⁶, G², T, Y¹, Y², n and p are as defined for formula I and E is hydrogen, a protecting group such as acetyl, benzyl or tert-butoxycarbonyl or a group M, can be obtained by transformation of a compound of formula VI, wherein R³, R⁴, R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with a compound of formula XI, wherein R¹⁶ is as defined for formula, and hydroxylamine and sodium hypochlorite. This is shown in Scheme 7.

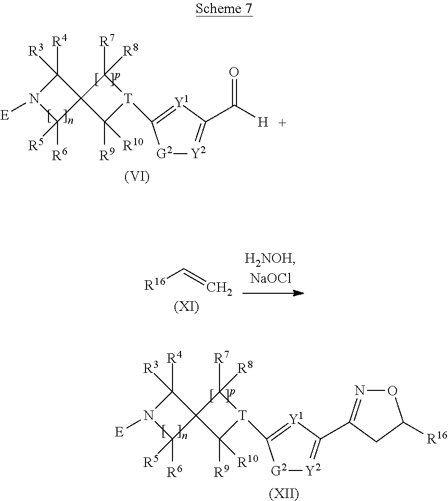

The compounds of formula XV, wherein R³, R⁴, R⁵, R⁶, R⁷, R⁸, R⁹, R¹⁰, R¹¹, G², Y¹, Y², n and p are as defined for formula I and E is hydrogen, a protecting group such as acetyl, benzyl or tert-butoxycarbonyl or a group M, can be obtained by transformation of a compound of formula XIII, wherein R³, R⁴, R⁵, R⁶, R⁷, R⁸, R⁹, R¹⁰, n and p are as defined for formula I and E is hydrogen, a protecting group such as acetyl, benzyl or tert-butoxycarbonyl or a group M, with a compound of formula XIV, wherein R¹¹, G², Y¹, Y² and Hal is halogen, preferably chloro, bromo or iodo, with a transition metal catalyst and a ligand or with a base. This is shown in Scheme 8.

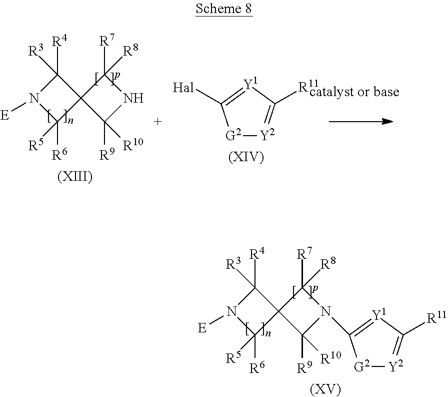

Surprisingly, it has now been found that the novel compounds of formula I have, for practical purposes, a very advantageous level of biological activity for protecting plants against diseases that are caused by fungi.

The compounds of formula I can be used in the agricultural sector and related fields of use e.g. as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula I can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula I as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula I before planting: seed, for example, can be dressed before being sown. The active ingredients according to the invention can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds according to present invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula I are, for example, effective against the phytopathogenic fungi of the following classes: Fungi imperfecti (e.g. Alternaria spp.), Basidiomycetes (e.g. Corticium spp., Ceratobasidium spp., Waltea spp., Thanatephorus spp., Rhizoctonia spp., Hemileia spp., Puccinia spp., Phakopsora spp., Ustilago spp., Tilletia spp.), Ascomycetes (e.g. Venturia spp., Blumeria spp., Erysiphe spp., Podosphaera spp., Uncinula spp., Monilinia spp., Sclerotinia spp., Colletotrichum spp., Glomerella spp., Fusarium spp., Gibberella spp., Monographella spp., Phaeosphaeria spp., Mycosphaerella spp., Cercospora spp., Pyrenophora spp., Rhynchosporium spp., Magnaporthe spp., Gaeumannomyces spp., Oculimacula spp., Ramularia spp., Botryotinia spp.) and Oomycetes (e.g. Phytophthora spp., Pythium spp., Plasmopara spp., Peronospora spp., Pseudoperonospora spp. Bremia spp). Outstanding activity is observed against downy mildew (e.g. Plasmopara viticola) and late blight (e.g. Phytophthora infestans). Furthermore, the novel compounds of formula I are effective against phytopathogenic gram negative and gram positive bacteria (e.g. Xanthomonas spp, Pseudomonas spp, Erwinia amylovora, Ralstonia spp.) and viruses (e.g. tobacco mosaic virus).

Within the scope of present invention, target crops and/or useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as turf and ornamentals.

The useful plants and or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 9533818, and EP-A-0 353 191. The methods of producing such transgenic plants are generally known to the person skilled in the art and are described, for example, in the publications mentioned above.

The term “locus” of a plant as used herein is intended to embrace the place on which the plants are growing, where the plant propagation materials of the plants are sown or where the plant propagation materials of the plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I are normally used in the form of compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula I may be used in the form of (fungicidal) compositions for controlling or protecting against phytopathogenic microorganisms, comprising as active ingredient at least one compound of formula I or of at least one preferred individual compound as above-defined, in free form or in agrochemically usable salt form, and at least one of the above-mentioned adjuvants.

The invention provides a composition, preferably a fungicidal composition, comprising at least one compound formula I an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally active compounds, for example an additional fungicidal active ingredient in addition to the compound of formula I.

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities. Examples of suitable additional active ingredients include the following: Azoxystrobin (131860-33-8), Dimoxystrobin (149961-52-4), Enestrobin (238410-11-2), Fluoxastrobin (193740-76-0), Kresoxim-methyl (143390-89-0), Metominostrobin (133408-50-1), Orysastrobin (248593-16-0), Picoxystrobin (117428-22-5), Pyraclostrobin (175013-18-0), trifloxystrobin (141517-21-7), Azaconazole (60207-31-0), Bromuconazole (116255-48-2), Cyproconazole (94361-06-5), Difenoconazole (119446-68-3), Diniconazole (83657-24-3), Diniconazole-M (83657-18-5), Epoxiconazole (13385-98-8), Fenbuconazole (114369-43-6), Fluquinconazole (136426-54-5), Flusilazole (85509-19-9), Flutriafol (76674-21-0), Hexaconazole (79983-71-4), Imazalil (58594-72-2), Imibenconazole (86598-92-7), Ipconazole (125225-28-7), Metconazole (125116-23-6), Myclobutanil (88671-89-0), Oxpoconazole (174212-12-5), Pefurazoate (58011-68-0), Penconazole (66246-88-6), Prochloraz (67747-09-5), Propiconazole (60207-90-1), Prothioconazole (178928-70-6), Simeconazole (149508-90-7), Tebuconazole (107534-96-3), Tetraconazole (112281-77-3), Triadimefon (43121-43-3), Triadimenol (55219-65-3), Triflumizole (99387-89-0), Triticonazole (131983-72-7), Diclobutrazol (76738-62-0), Etaconazole (60207-93-4), Fluconazole (86386-73-4), Fluconazole-cis (112839-32-4), Thiabendazole (148-79-8), Quinconazole (103970-75-8), Fenpiclonil (74738-17-3), Fludioxonil (131341-86-1), Cyprodinil (121552-61-2), Mepanipyrim (110235-47-7), Pyrimethanil (53112-28-0), Aldimorph (91315-15-0), Dodemorph (1593-77-7), Fenpropimorph (67564-91-4), Tridemorph (81412-43-3), Fenpropidin (67306-00-7), Spiroxamine (118134-30-8), Isopyrazam (881685-58-1), Sedaxane (874967-67-6), Bixafen (581809-46-3), Penthiopyrad (183675-82-3), Fluxapyroxad (907204-31-3), Boscalid (188425-85-6), Penflufen (494793-67-8), Fluopyram (658066-35-4), Mandipropamid (374726-62-2), Benthiavalicarb (413615-35-7), Dimethomorph (110488-70-5), Chlorothalonil (1897-45-6), Fluazinam (79622-59-6), Dithianon (3347-22-6), Metrafenone (220899-03-6), Tricyclazole (41814-78-2), Mefenoxam (70630-17-0), Metalaxyl (57837-19-1), Acibenzolar (126448-41-7) (Acibenzolar-S-methyl (126448-41-7)), Mancozeb (8018-01-7), Ametoctradine (865318-97-4) Cyflufenamid (180409-60-3), and Kresoxim-methyl (143390-89-0), Ipconazole (125225-28-7), Amisulbrom (348635-87-0), Cyflufenamid (180409-60-3), Ethaboxam (16650-77-3), Fluopicolide (239110-15-7), Fluthianil (304900-25-2), Isotianil (224049-04-1), Proquinazid (189278-12-4), Valiphenal (283159-90-0), 1-methyl-cyclopropene (3100-04-7), Trifloxystrobin (141517-21-7), Sulfur (7704-34-9), Copper ammoniumcarbonate (CAS 33113-08-5); Copper oleate (CAS 1120-44-1); Folpet (133-07-3), Quinoxyfen (124495-18-7), Captan (133-06-2), Fenhexamid (126833-17-8), Glufosinate and its salts (51276-47-2, 35597-44-5 (S-isomer)), Glyphosate (1071-83-6) and its salts (69254-40-6 (Diammonium), 34494-04-7 (Dimethylammonium), 38641-94-0 (Isopropylammonium), 40465-66-5 (Monoammonium), 70901-20-1 (Potassium), 70393-85-0 (Sesquisodium), 81591-81-3 (Trimesium)), 1-methyl-3-difluoromethyl-1H-pyrazole-4-carboxylic acid (2-dichloromethylene-3-ethyl-1-methyl-indan-4-yl)-amide (1072957-71-1), 1-methyl-3-difluoromethyl-1H-pyrazole-4-carboxylic acid (4′-methylsulfanyl-biphenyl-2-yl)-amide, 1-methyl-3-difluoromethyl-4H-pyrazole-4-carboxylic acid [2-(2,4-dichloro-phenyl)-2-methoxy-1-methyl-ethyl]amide, (5-Chloro-2,4-dimethyl-pyridin-3-yl)-(2,3,4-trimethoxy-6-methyl-phenyl)-methanone, (5-Bromo-4-chloro-2-methoxy-pyridin-3-yl)-(2,3,4-trimethoxy-6-methyl-phenyl)-methanone, 2-{2-[(E)-3-(2,6-Dichloro-phenyl)-1-methyl-prop-2-en-(E)-ylideneaminooxymethyl]-phenyl}-2-[(Z)-methoxyimino]-N-methyl-acetamide, 3-[5-(4-Chloro-phenyl)-2,3-dimethyl-isoxazolidin-3-yl]-pyridine.

Another aspect of invention is related to the use of a compound of formula I or of a preferred individual compound as above-defined, of a composition comprising at least one compound of formula I or at least one preferred individual compound as above-defined, or of a fungicidal mixture comprising at least one compound of formula I or at least one preferred individual compound as above-defined, in admixture with other fungicides, as described above, for controlling or preventing infestation of plants, e.g. useful plants such as crop plants, propagation material thereof, e.g. seeds, harvested crops, e.g. harvested food crops, or non-living materials by phytopathogenic microorganisms, preferably fungal organisms.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula I or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a compound of formula I, or an agrochemical composition which contains at least one of said compounds, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, e.g. a composition containing the compound of formula I, and, if desired, a solid or liquid adjuvant or monomers for encapsulating the compound of formula I, may be prepared in a known manner, typically by intimately mixing and/or grinding the compound with extenders, for example solvents, solid carriers and, optionally, surface active compounds (surfactants).

The agrochemical formulations and/or compositions will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

Whereas it is preferred to formulate commercial products as concentrates, the end user will normally use dilute formulations.

The following non-limiting examples illustrate the above-described invention in more detail.

### EXAMPLE 1

This example illustrates the preparation of 2-[3,5-bis(difluoromethyl)pyrazol-1-yl]-1-[2-[4-[5-(2,6-difluorophenyl)-4,5-dihydroisoxazol-3-yl]thiazol-2-yl]-2,6-diazaspiro[3.3]heptan-6-yl]ethanone (Compound No. I.az.022)

### a) Preparation of 2-bromothiazole-4-carbaldehyde oxime

2-bromothiazole-4-carbaldehyde (4.50 g, 4.50 g, 23.4 mmol) was dissolved in EtOH (100 mL), followed by addition of hydroxylamine 50% aq. solution (6.19 g, 93.7 mmol) stirred for 1.5 h at 60° C. Solvent was then evaporated and the residue was dissolved with ethylacetate and washed with water, 0.2M HCl and brine to give 2-bromothiazole-4-carbaldehyde oxime (mixture of isomers 4.72 g, 97%). Mixture of isomers ¹H-NMR (400 MHz, CDCl₃): δ=7.5 (s, 1H), 7.8 (s, 1H), 8.2 (s, 1H), 8.4 (s, 1H), 8.8 (br, 1H), 10.0 (br, 1H). MS: mz=208 (M+1).

### b) Preparation of 3-(2-bromothiazol-4-yl)-5-(2,6-difluorophenyl)-4,5-dihydroisoxazole

To a solution of 2-bromothiazole-4-carbaldehyde oxime (0.250 g, 1.21 mmol) in THF (10 mL) was added 2,6-dfluorostyrene (0.256 g, 1.81 mmol) following by dropwise addition of the sodium hypochlorite (1.59 g, 3.62 mmol). After stirring 3 h min at room temperature, the reaction mixture was diluted with ethylacetate, then the organic phase was separated. The aqueous phase was extracted 3× with ethylacetate. The organic phases were combined, washed with brine and concentrated to give 3-(2-bromothiazol-4-yl)-5-(2,6-difluorophenyl)-4,5-dihydroisoxazole (311 mg, 75%). ¹H-NMR (400 MHz, CDCl₃): δ=3.55-3.70 (m, 1H), 3.75-3.90 (m, 1H), 6.05-6.15 (m, 1H), 6.85-7-00 (m, 2H), 7.2-7.4 (m, 1H), 7.7 (s, 1H). MS: mz=346 (M+1).

### c) Preparation of tert-butyl 2-[4-[5-(2,6-difluorophenyl)-4,5-dihydroisoxazol-3-yl]thiazol-2-yl]-2,6-diazaspiro[3.3]heptane-6-carboxylate

To a solution of tert-butyl 6-aza-2-azoniaspiro[3.3]heptane-6-carboxylate oxalate (0.956 mmol) in acetonitrile (20 mL) were added dipotassium carbonic acid (0.670 g, 4.78 mmol) then 3-(2-bromothiazol-4-yl)-5-(2,6-difluorophenyl)-4,5-dihydroisoxazole (0.330 g, 0.956 mmol). After heating at reflux for 5 days, solvent was evaporated and the residue was suspended in ethylacetate, washed with water 2× and brine, dried and solvent evaporated under pressure. The crude mixture was purified by column chromatography (ethylacetate/cyclohexane, 0-50%) to give tert-butyl 2-[4-[5-(2,6-difluorophenyl)-4,5-dihydroisoxazol-3-yl]thiazol-2-yl]-2,6-diazaspiro[3.3]heptane-6-carboxylate (371 mg, 84%). ¹H-NMR (400 MHz, CDCl₃): δ=1.48 (s, 9H), 3.50-3.60 (m, 1H), 3.65-3.75 (m, 1H), 4.11 (s, 4H), 4.25 (s, 4H), 6.00-6.10 (m, 1H), 6.85-6.98 (m, 2H), 7-00 (s, 1H), 7.25-7.4 (m, 1H), 7.7 (s, 1H). MS: mz=463 (M+1).

### d) Preparation of 3-[2-(2,6-diazaspiro[3.3]heptan-6-yl)thiazol-4-yl]-5-(2,6-difluorophenyl)-4,5-dihydroisoxazole hydrochloride

To a solution of tert-butyl 2-[4-[5-(2,6-difluorophenyl)-4,5-dihydroisoxazol-3-yl]thiazol-2-yl]-2,6-diazaspiro[3.3]heptane-6-carboxylate in MeOH (0.216 mL) was added 4M HCl (0.432 mmol, 0.108 mL) at 0° C. After stirring at room temperature overnight, solvent was evaporated, to give 3-[2-(2,6-diazaspiro[3.3]heptan-6-yl)thiazol-4-yl]-5-(2,6-difluorophenyl)-4,5-dihydroisoxazole hydrochloride (80 mg, 93%). ¹H-NMR (400 MHz, CDCl₃): δ=3.45-3.55 (m, 1H), 3.60-3.70 (m, 1H), 3.80 (s, 4H), 3.90 (s, 4H), 5.90-6.10 (m, 1H), 6.80-7.00 (m, 3H), 7.20-7.35 (m, 1H). MS: mz=436 (M+1).

### e) 2-3,5-bis(difluoromethyl)pyrazol-1-yl]-1-[2-[4-[5-(2,6-difluorophenyl)-4,5-dihydroisoxazol-3-yl]thiazol-2-yl]-2,6-diazaspiro[3.3]heptan-6-yl]ethanone (I.az.022)

To a solution of 2-[3,5-bis(difluoromethyl)pyrazol-1-yl]acetic acid (0.014 g, 0.061 mmol) in acetonitrile (3 mL) was added triethylamine (0.026 mL) then 1-ethyl-3-(3-dimethylaminopropyl)carbodiimide) (0.026 g, 0.13 mmol) then 1-hydroxy-7-azabenzotriazole (0.018 g, 0.13 mmol). After stirring 10 min at rt, a solution of 3-[2-(2,6-diazaspiro[3.3]heptan-6-yl)thiazol-4-yl]-5-(2,6-difluorophenyl)-4,5-dihydroisoxazole hydrochloride (0.022 g, 0.061 mmol) in DMF (0.6 mL) was added. After stirring overnight at room temperature, solvent was evaporated and the residue was dissolved in ethylacetate, washed with water, sodium-bicarbonate, and brine. Organic phase was then dried and evaporated to give 2-[3,5-bis(difluoromethyl)pyrazol-1-yl]-1-[2-[4-[5-(2,6-difluorophenyl)-4,5-dihydroisoxazol-3-yl]thiazol-2-yl]-2,6-diazaspiro[3.3]heptan-6-yl]ethanone (I.az.022) (14 mg, 40%). ¹H-NMR (400 MHz, CDCl₃): δ=3.42-3.52 (m, 1H), 3.58-3.70 (m, 1H), 4.15 (s, 2H), 4.25 (s, 4H), 4.30 (s, 2H), 4.82 (s, 2H), 5.95-6.05 (m, 1H) 6.40-7.05 (m, 6H), 7.20-7.30 (m, 1H). MS: mz=571 (M+1).

### EXAMPLE 2

This example illustrates the preparation of 1-[2-[4-[5-(2,6-difluorophenyl)-4,5-dihydroisoxazol-3-yl]thiazol-2-yl]-2,6-diazaspiro[3.3]heptan-6-yl]-2-[3-methyl-5-(trifluoromethyl)pyrazol-1-yl]ethanone (Compound No. I.az.004)

To a solution of 1H-Pyrazole-1-acetic acid, 5-methyl-3-(trifluoromethyl) (0.032 g, 0.15 mmol) in acetonitrile (0.3 mL) was added triethylamine (0.046 g), then 1-ethyl-3-(3-dimethylaminopropyl)carbodiimide) (0.035 g, 0.18 mmol) and 1-hydroxy-7-azabenzotriazole (0.025 g, 0.18 mmol). After stirring 10 min. at room temperature, 3-[2-(2,6-diazaspiro[3.3]heptan-6-yl)thiazol-4-yl]-5-(2,6-difluorophenyl)-4,5-dihydroisoxazole hydrochloride (0.055 g, 0.15 mmol) was added. After stirring overnight at room temperature, solvent was evaporated and the residue was dissolved in ethylacetate, washed with water, sodium-bicarbonate, 0.5M HCl, and brine. Organic phase was then dried and solbent were evaporated under reduced pressure. The crude mixture was purified by column chromatography (ethylacetate/cyclohexane, 0-50%) to give 1-[2-[4-[5-(2,6-difluorophenyl)-4,5-dihydroisoxazol-3-yl]thiazol-2-yl]-2,6-diazaspiro[3.3]heptan-6-yl]-2-[3-methyl-5-(trifluoromethyl)pyrazol-1-yl]ethanone (Compound No. I.az.004) (12 mg, 15%). ¹H-NMR (400 MHz, CDCl₃): δ=2.25 (s, 3H), 3.39.3.50 (m, 1H), 3.55-3.62 (m, 1H), 4.15 (s, 8H), 4.70 (s, 2H), 5.90-6.00 (m, 1H), 6.25 (s, 1H), 6.75-6.90 (m, 2H), 7.14 (s, 1H), 7.15-7.25 (m, 1H). MS: m/z=553 (M+1).

Table 1 below illustrates examples of individual compounds of formula I according to the invention.

TABLE 1
individual compounds of formula I according to the invention

| Comp. | | | | | | |
|---|---|---|---|---|---|---|
| No. | R¹ | R² | T | Y¹ | G² | Y² |
| 01 | F₃C | H₃C | CH | N | S | CH |
| 02 | F₃C | H₃C | CH | N | S | N |
| 03 | F₃C | H₃C | CH | N | O | CH |
| 04 | F₃C | H₃C | N | N | S | CH |
| 05 | F₃C | H₃C | N | N | S | N |
| 06 | F₃C | H₃C | N | N | O | CH |
| 07 | H₃C | H₃C | CH | N | S | CH |
| 08 | H₃C | H₃C | CH | N | S | N |
| 09 | H₃C | H₃C | CH | N | O | CH |
| 10 | H₃C | H₃C | N | N | S | CH |
| 11 | H₃C | H₃C | N | N | S | N |
| 12 | H₃C | H₃C | N | N | O | CH |
| 13 | F₂HC | H₃C | CH | N | S | CH |
| 14 | F₂HC | H₃C | CH | N | S | N |
| 15 | F₂HC | H₃C | CH | N | O | CH |
| 16 | F₂HC | H₃C | N | N | S | CH |
| 17 | F₂HC | H₃C | N | N | S | N |
| 18 | F₂HC | H₃C | N | N | O | CH |
| 19 | F₂HC | F₂HC | CH | N | S | CH |
| 20 | F₂HC | F₂HC | CH | N | S | N |
| 21 | F₂HC | F₂HC | CH | N | O | CH |
| 22 | F₂HC | F₂HC | N | N | S | CH |
| 23 | F₂HC | F₂HC | N | N | S | N |
| 24 | F₂HC | F₂HC | N | N | O | CH |
| 25 | F₃C | F₃C | CH | N | S | CH |
| 26 | F₃C | F₃C | CH | N | S | N |
| 27 | F₃C | F₃C | CH | N | O | CH |
| 28 | F₃C | F₃C | N | N | S | CH |
| 29 | F₃C | F₃C | N | N | S | N |
| 30 | F₃C | F₃C | N | N | O | CH |

where
a) 30 compounds of formula (I.a):

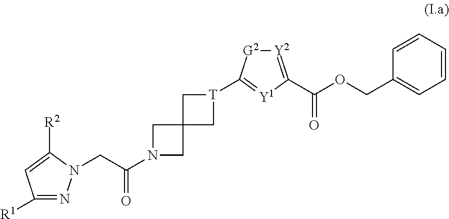

wherein R¹, R², G², T, Y¹ and Y² are as defined in Table 1.
b) 30 compounds of formula (I.b):

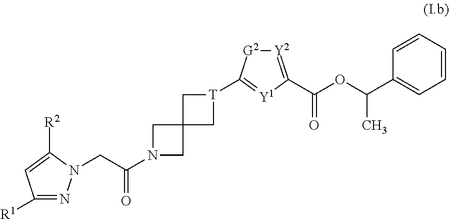

wherein R¹, R², G², T, Y¹ and Y² are as defined in Table 1.
c) 30 compounds of formula (I.c):

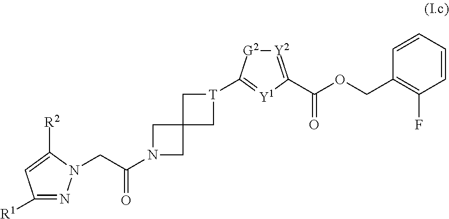

wherein R¹, R², G², T, Y¹ and Y² are as defined in Table 1.
d) 30 compounds of formula (I.d):

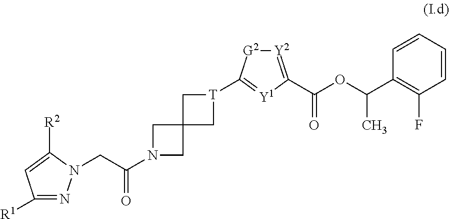

Wherein R¹, R², G², T, Y¹ and Y² are as defined in Table 1.
e) 30 compounds of formula (I.e):

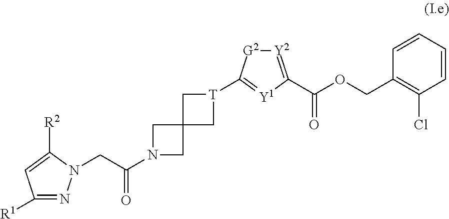

wherein R¹, R², G², T, Y¹ and Y² are as defined in Table 1.
f) 30 compounds of formula (I.f):

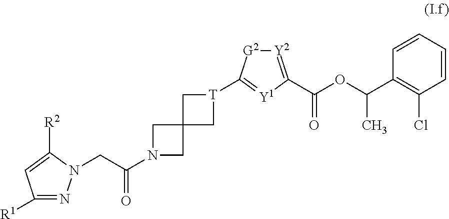

wherein R¹, R², G², T, Y¹ and Y² are as defined in Table 1.
g) 30 compounds of formula (I.g):

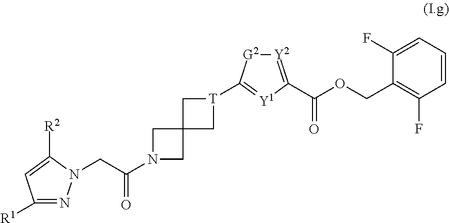

wherein R¹, R², G², T, Y¹ and Y² are as defined in Table 1.
h) 30 compounds of formula (I.h):

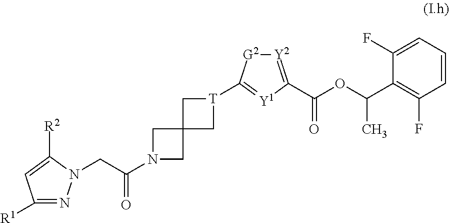

wherein R¹, R², G², T, Y¹ and Y² are as defined in Table 1.
i) 30 compounds of formula (I.i):

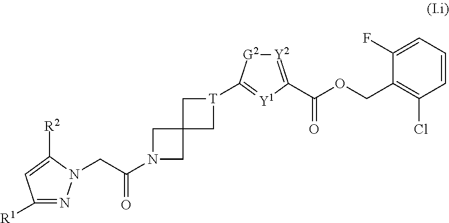

wherein R¹, R², G², T, Y¹ and Y² are as defined in Table 1.
j) 30 compounds of formula (I.j):

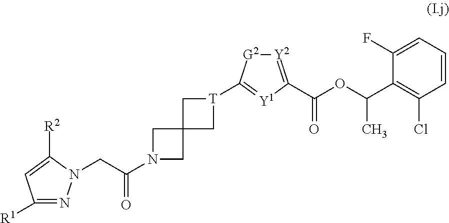

wherein R¹, R², G², T, Y¹ and Y² are as defined in Table 1.
k) 30 compounds of formula (I.k):

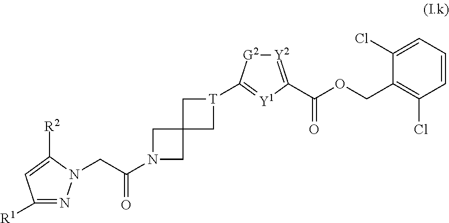

wherein R¹, R², G², T, Y¹ and Y² are as defined in Table 1.
m) 30 compounds of formula (I.m):

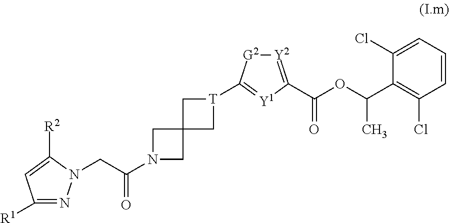

wherein R¹, R², G², T, Y¹ and Y² are as defined in Table 1.
n) 30 compounds of formula (I.n):

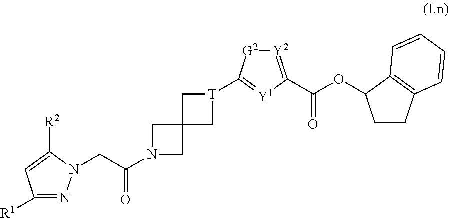

wherein R¹, R², G², T, Y¹ and Y² are as defined in Table 1.
o) 30 compounds of formula (I.o):

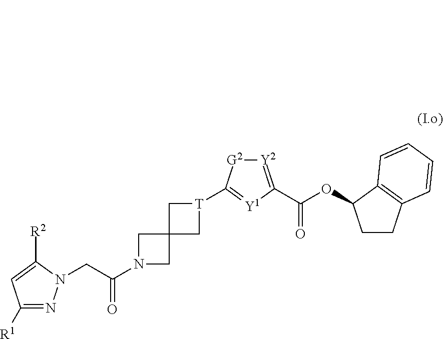

wherein R¹, R², G², T, Y¹ and Y² are as defined in Table 1.
p) 30 compounds of formula (I.p):

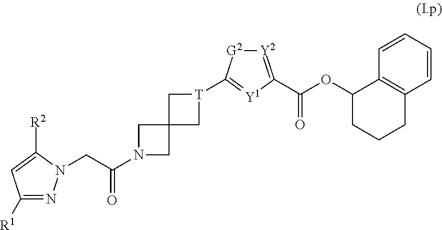

wherein R¹, R², G², T, Y¹ and Y² are as defined in Table 1.
q) 30 compounds of formula (I.q):

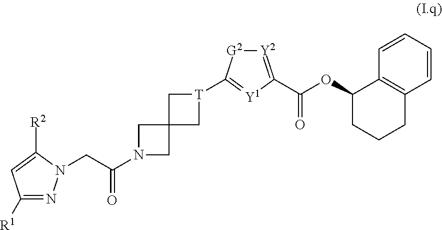

wherein R¹, R², G², T, Y¹ and Y² are as defined in Table 1.
r) 30 compounds of formula (I.r):

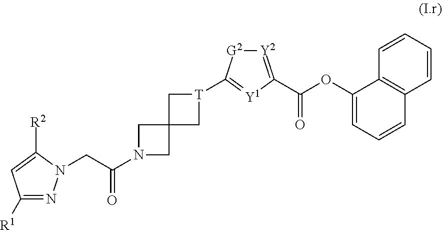

wherein R¹, R², G², T, Y¹ and Y² are as defined in Table 1.
s) 30 compounds of formula (I.s):

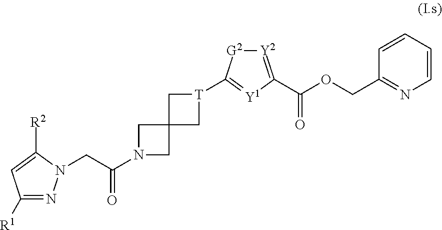

wherein R¹, R², G², T, Y¹ and Y² are as defined in Table 1.
t) 30 compounds of formula (I.t):

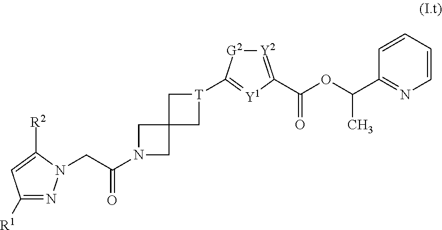

wherein R¹, R², G², T, Y¹ and Y² are as defined in Table 1.
u) 30 compounds of formula (I.u):

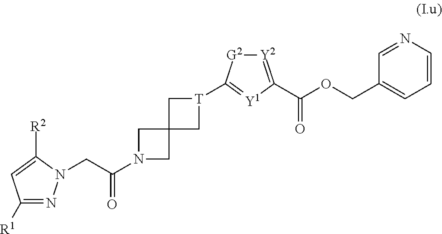

wherein R¹, R², G², T, Y¹ and Y² are as defined in Table 1.
v) 30 compounds of formula (I.v):

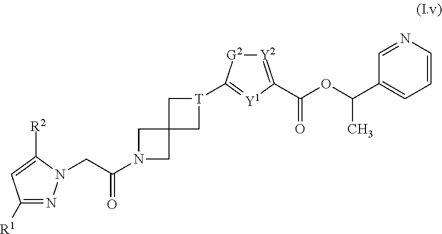

wherein R¹, R², G², T, Y¹ and Y² are as defined in Table 1.
w) 30 compounds of formula (I.w):

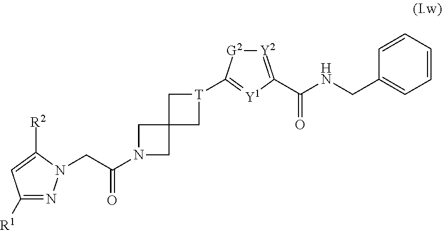

wherein R¹, R², G², T, Y¹ and Y² are as defined in Table 1.
x) 30 compounds of formula (I.x):

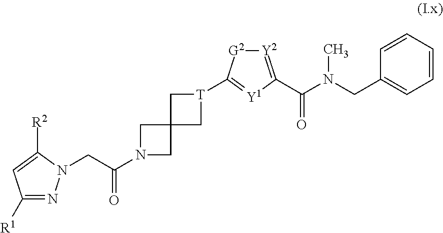

wherein R¹, R², G², T, Y¹ and Y² are as defined in Table 1.
y) 30 compounds of formula (I.y):

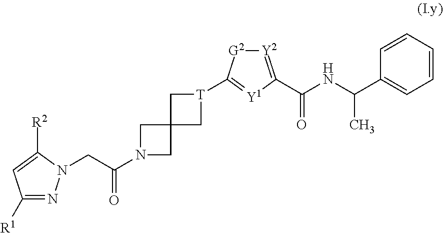

wherein R¹, R², G², T, Y¹ and Y² are as defined in Table 1.
z) 30 compounds of formula (I.z):

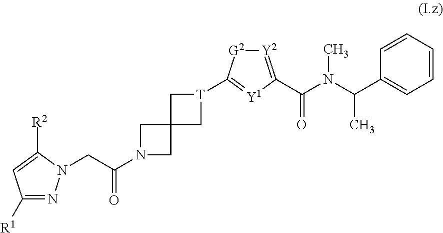

wherein R¹, R², G², T, Y¹ and Y² are as defined in Table 1.
aa) 30 compounds of formula (I.aa):

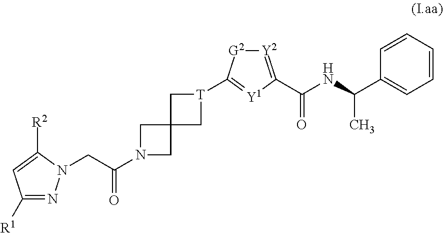

wherein R¹, R², G², T, Y¹ and Y² are as defined in Table 1.
ab) 30 compounds of formula (I.ab):

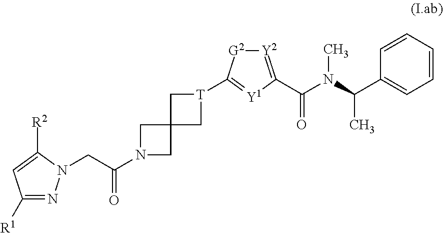

wherein R¹, R², G², T, Y¹ and Y² are as defined in Table 1.
ac) 30 compounds of formula (I.ac):

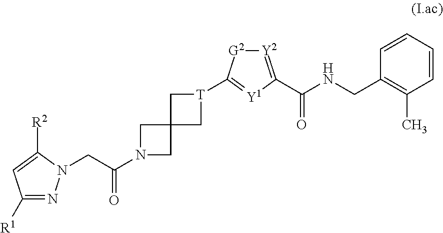

wherein R¹, R², G², T, Y¹ and Y² are as defined in Table 1.
ad) 30 compounds of formula (I.ad):

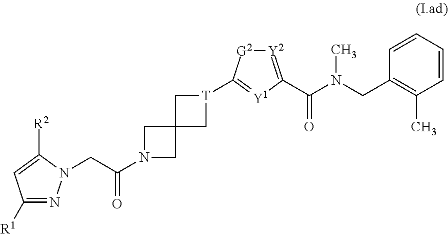

wherein R¹, R², G², T, Y¹ and Y² are as defined in Table 1.
ae) 30 compounds of formula (I.ae):

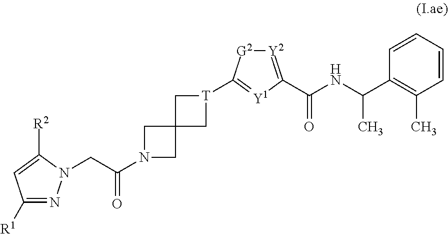

wherein R¹, R², G², T, Y¹ and Y² are as defined in Table 1.
af) 30 compounds of formula (I.af):

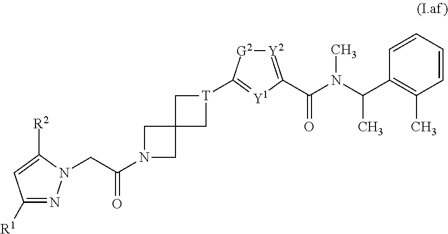

wherein R¹, R², G², T, Y¹ and Y² are as defined in Table 1.
ag) 30 compounds of formula (I.ag):

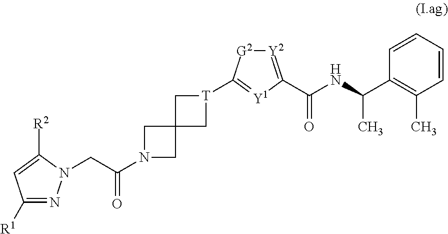

wherein R¹, R², G², T, Y¹ and Y² are as defined in Table 1.
ah) 30 compounds of formula (I.ah):

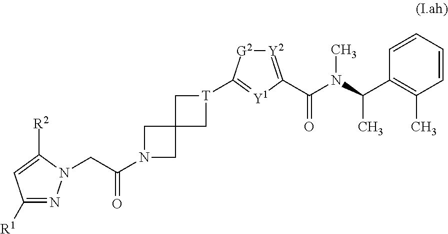

wherein R¹, R², G², T, Y¹ and Y² are as defined in Table 1.
ai) 30 compounds of formula (I.ai):

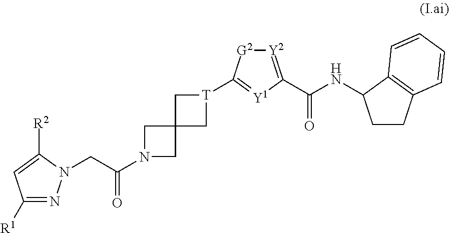

wherein R¹, R², G², T, Y¹ and Y² are as defined in Table 1.
aj) 30 compounds of formula (I.aj):

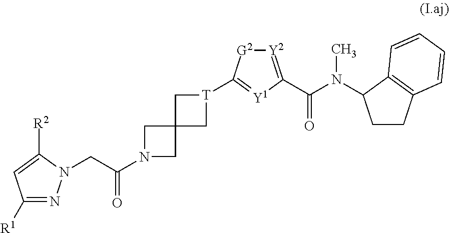

wherein R¹, R², G², T, Y¹ and Y² are as defined in Table 1.
ak) 30 compounds of formula (I.ak):

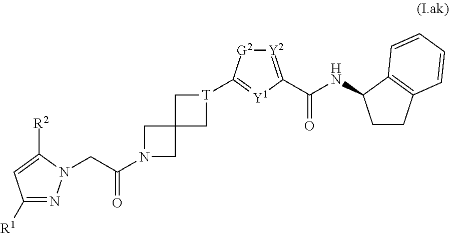

wherein R¹, R², G², T, Y¹ and Y² are as defined in Table 1.
am) 30 compounds of formula (I.am):

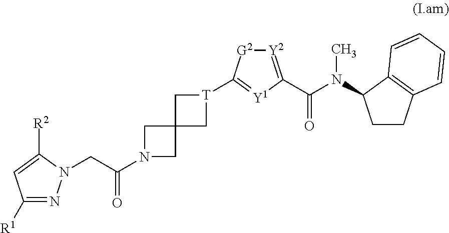

wherein R¹, R², G², T, Y¹ and Y² are as defined in Table 1.
an) 30 compounds of formula (I.an):

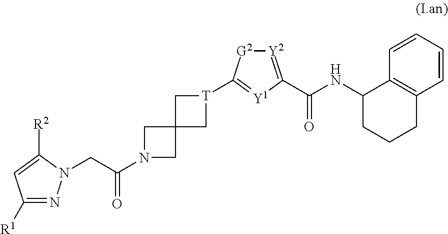

wherein R¹, R², G², T, Y¹ and Y² are as defined in Table 1.
ao) 30 compounds of formula (I.ao):

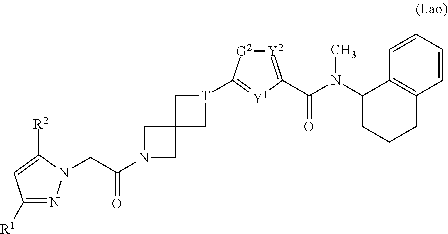

wherein R¹, R², G², T, Y¹ and Y² are as defined in Table 1.
ap) 30 compounds of formula (I.ap):

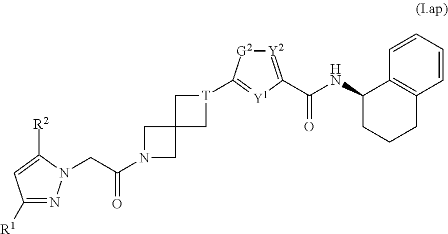

wherein R¹, R², G², T, Y¹ and Y² are as defined in Table 1.
aq) 30 compounds of formula (I.aq):

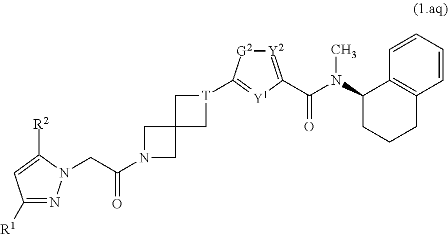

wherein R¹, R², G², T, Y¹ and Y² are as defined in Table 1.
ar) 30 compounds of formula (I.ar):

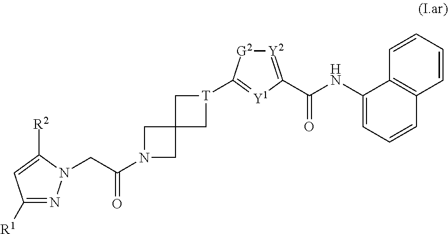

wherein R¹, R², G², T, Y¹ and Y² are as defined in Table 1.
as) 30 compounds of formula (I.as):

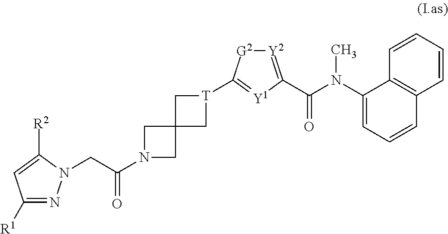

wherein R¹, R², G², T, Y¹ and Y² are as defined in Table 1.
at) 30 compounds of formula (I.at):

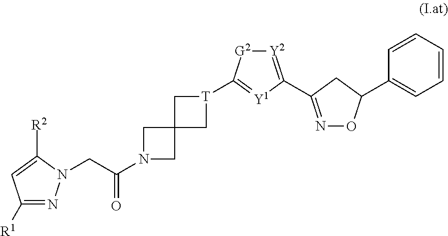

wherein R¹, R², G², T, Y¹ and Y² are as defined in Table 1.
au) 30 compounds of formula (I.au):

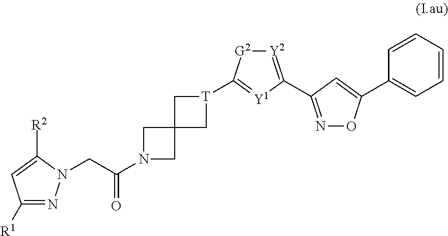

wherein R¹, R², G², T, Y¹ and Y² are as defined in Table 1.
av) 30 compounds of formula (I.av):

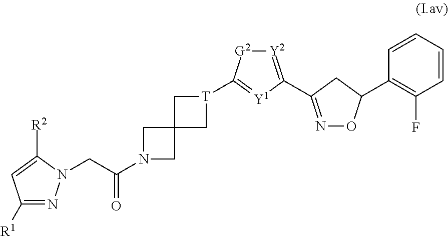

wherein R¹, R², G², T, Y¹ and Y² are as defined in Table 1.
aw) 30 compounds of formula (I.aw):

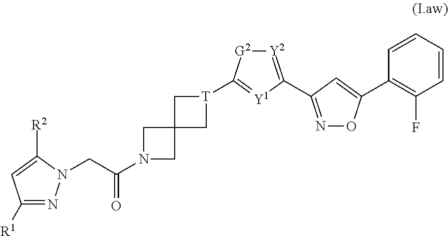

wherein R¹, R², G², T, Y¹ and Y² are as defined in Table 1.
ax) 30 compounds of formula (I.ax):

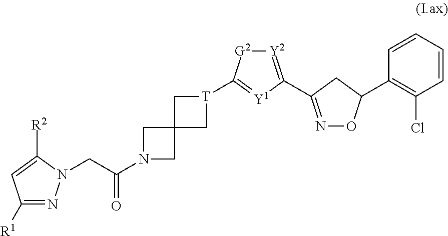

wherein R¹, R², G², T, Y¹ and Y² are as defined in Table 1.
ay) 30 compounds of formula (Lay):

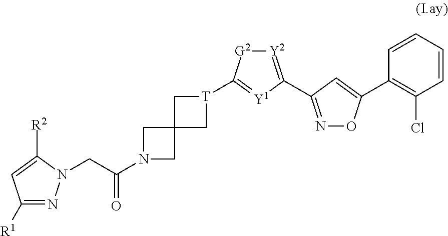

wherein R¹, R², G², T, Y¹ and Y² are as defined in Table 1.
az) 30 compounds of formula (I.az):

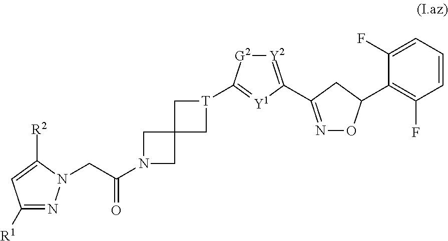

wherein R¹, R², G², T, Y¹ and Y² are as defined in Table 1.
ba) 30 compounds of formula (I.ba):

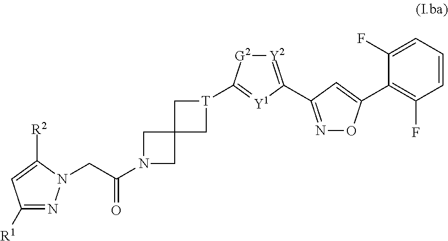

wherein R¹, R², G², T, Y¹ and Y² are as defined in Table 1.
bb) 30 compounds of formula (I.bb):

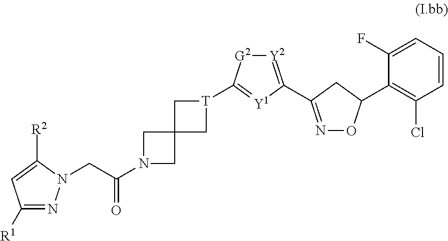

wherein R¹, R², G², T, Y¹ and Y² are as defined in Table 1.
bc) 30 compounds of formula (I.bc):

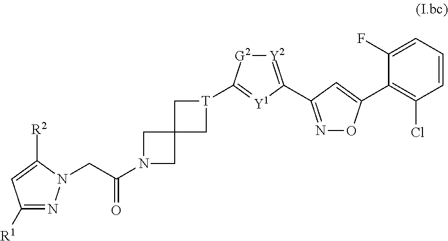

wherein R¹, R², G², T, Y¹ and Y² are as defined in Table 1.
bd) 30 compounds of formula (I.bd):

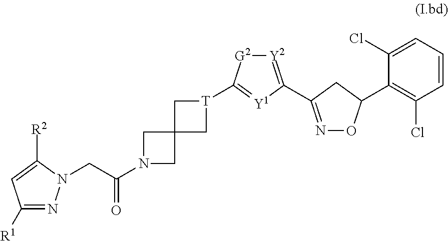

wherein R¹, R², G², T, Y¹ and Y² are as defined in Table 1.
be) 30 compounds of formula (I.be):

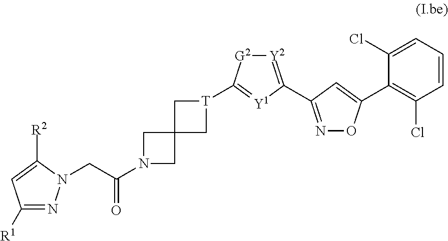

wherein R¹, R², G², T, Y¹ and Y² are as defined in Table 1.
bf) 30 compounds of formula (I.bf):

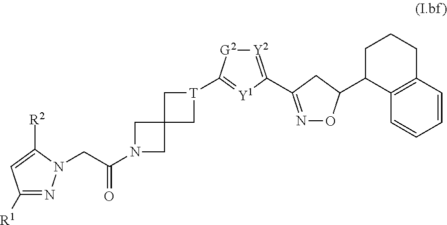

wherein R¹, R², G², T, Y¹ and Y² are as defined in Table 1.

The compounds according to the present invention can be prepared according to the above-mentioned reaction schemes, in which, unless otherwise stated, the definition of each variable is as defined above for a compound of formula (I).

TABLE 2
Melting point and LC/MS data for compounds of Table 1

| | | |
|---|---|---|
| | Melting point | |
| Compound No. | (° C.) | LC/MS |

| | |
|---|---|
| I.az.004 | Rt = 0.98 min; MS: m/z = 553 (M + 1) |
| I.az.022 | Rt = 0.97 min; MS: m/z = 571 (M + 1) |

### BIOLOGICAL EXAMPLES

### Phytophthora infestansTomato/Leaf Disc Preventative (Tomato Late Blight)

Tomato leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 16° C. and 75% rh under a light regime of 24 h darkness followed by 12 h light/12 h darkness in a climate cabinet and the activity of a compound is assessed as percent disease control compared to untreated when an appropriate level of disease damage appears in untreated check leaf disks (5-7 days after application).

Compounds I.az.04, I.az.022 at 200 ppm gives at least 70% disease control in this test when compared to untreated control leaf discs under the same conditions, which show extensive disease development.

Phytophthora infestans/Potato/Preventative (Potato Late Blight)

2-week old potato plants cv. Bintje are sprayed in a spray chamber with the formulated test compound diluted in water. The test plants are inoculated by spraying them with a sporangia suspension 2 days after application. The inoculated test plants are incubated at 18° C. with 14 h light/day and 100% rh in a growth chamber and the percentage leaf area covered by disease is assessed when an appropriate level of disease appears on untreated check plants (5-7 days after application).

Phytophthora infestans/Potato/Curative (Potato Late Blight)

2-week old potato plants cv. Bintje are inoculated by spraying them with a sporangia suspension one day before application. The inoculated plants are sprayed in a spray chamber with the formulated test compound diluted in water. The inoculated test plants are incubated at 18° C. with 14 h light/day and 100% rh in a growth chamber and the percentage leaf area covered by disease is assessed when an appropriate level of disease appears on untreated check plants (3-4 days after application).

Phytophthora infestans/Potato Long Lasting (Potato Late Blight)

2-week old potato plants cv. Bintje are sprayed in a spray chamber with the formulated test compound diluted in water. The test plants are inoculated by spraying them with a sporangia suspension 6 days after application. The inoculated test plants are incubated at 18° C. with 14 h light/day and 100% rh in a growth chamber and the percentage leaf area covered by disease is assessed when an appropriate level of disease appears on untreated check plants (9-11 days after application).

Plasmopara viticola/Grape/Leaf Disc Preventative (Grape Downy Mildew)

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% rh under a light regime of 12 h light/12 h darkness in a climate cabinet and the activity of a compound is assessed as percent disease control compared to untreated when an appropriate level of disease damage appears in untreated check leaf disks (6-8 days after application).

Plasmopara viticola/Grape/Preventative (Drape Downy Mildew)

5-week old grape seedlings cv. Gutedel are sprayed in a spray chamber with the formulated test compound diluted in water. The test plants plants are inoculated by spraying a sporangia suspension on their lower leaf surface one day after application. The inoculated test plants are incubated at 22° C. and 100% rh in a greenhouse and the percentage leaf area covered by disease is assessed when an appropriate level of disease appears on untreated check plants (6-8 days after application).

Plasmopara viticola/Grape/Curative (Grape Downy Mildew)

5-week-old grape seedlings cv. Gutedel are inoculated by spraying a sporangia suspension on their lower leaf surface one day before application. The inoculated grape plants are sprayed in a spray chamber with the formulated test compound diluted in water. The inoculated test plants are incubated at 22° C. and 100% rh in a greenhouse and the percentage leaf area covered by disease is assessed when an appropriate level of disease appears on untreated check plants (4-6 days after application).

Plasmopara viticola/Grape/Long Lasting (Grape Downy Mildew)

5-week old grape seedlings cv. Gutedel are sprayed in a spray chamber with the formulated test compound diluted in water. The test plants are inoculated by spraying a sporangia suspension on their lower leaf surface 6 days after application. The inoculated test plants are incubated at 22° C. and 100% rh in a greenhouse and the percentage leaf area covered by disease is assessed when an appropriate level of disease appears on untreated check plants (11-13 days after application).

Pythium ultimum/Liquid Culture (Seedling Damping Off)

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added. The test plates are incubated at 24° C. and the inhibition of growth is determined photometrically 2-3 days after application.

1. A compound of formula I: 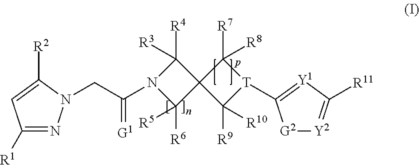
wherein,
G¹ and G² are independently O or S;
T is CR¹² or N;
Y¹ and Y² are independently CR¹³ or N;
n is 1 or 2;
p is 1 or 2,
R¹ and R² each independently are C₁-C₄alkyl, C₃-C₅cycloalkyl or C₁-C₄haloalkyl;
R³, R⁴, R⁵, R⁶, R⁷, R⁸, R⁹ and R¹⁹ each independently are hydrogen, halogen, cyano, hydroxyl, C₁-C₄alkyl, C₁-C₄haloalkyl and C₁-C₄alkoxy;
R¹¹ is C(═O)—OR¹⁴, C(═O)—NR¹⁴R¹⁵ or a 4- to 15-membered mono-, bi- or tricyclic, saturated, partially unsaturated or aromatic heterocyclic ringsystem optionally substituted by one or more R¹⁶;
R¹² is hydrogen, halogen or hydroxyl;
R¹³ is hydrogen, halogen or cyano;
R¹⁴ is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁶;
R¹⁵ is hydrogen, C₁-C₄alkyl, C₃-C₅cycloalkyl or C₁-C₄alkoxy;
each R¹⁶ independently is halogen, cyano, amino, nitro, hydroxyl, mercapto, C₁-C₈alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkoxy, C₃-C₈cycloalkyl-C₁-C₄alkylthio, C₁-C₈alkoxy, C₃-C₈cycloalkoxy, C₁-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₄alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁷; and
each R¹⁷ independently is halogen, cyano, C₁-C₄alkyl, C₁-C₄haloalkyl, C₁-C₄alkoxy or C₁-C₄haloalkoxy; or a salt or a N-oxide thereof.

2. A compound according to claim 1, wherein the compound of formula I is a compound of formula I-a 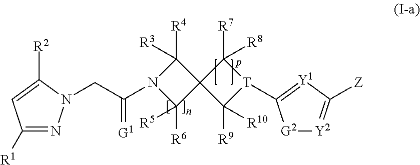
wherein Z is selected from Z-1 to Z-12 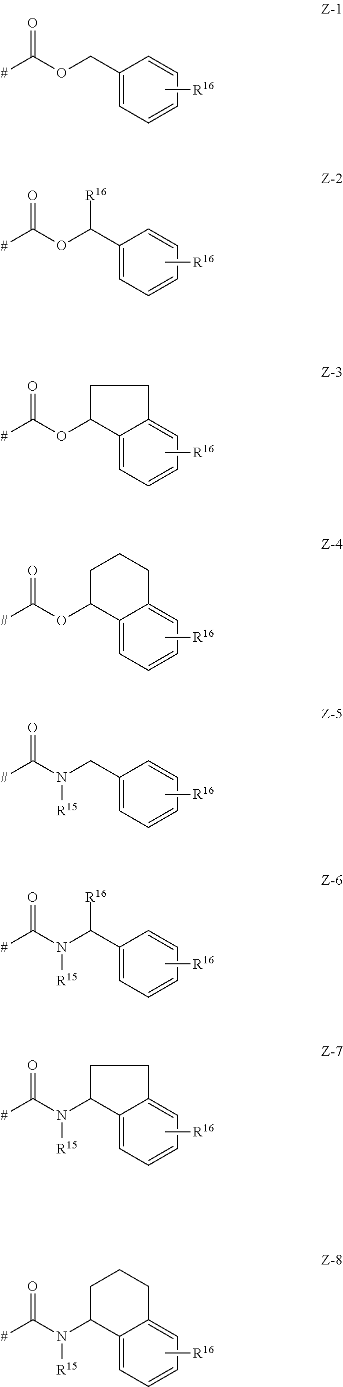 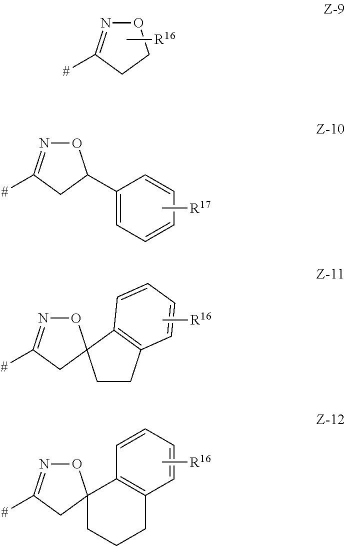
and G¹, G², T, Y¹, Y², n, p, R¹, R², R³, R⁴, R⁵, R⁶, R⁷, R⁸, R⁹, R¹⁰, R¹⁵, R¹⁶ and R¹⁷ are as defined for compound of formula I in claim 1.

3. A compound according to claim 2, wherein Z is selected from Z-1, Z-4, Z-8, Z-10 and Z-12.

4. A compound according to claim 2, wherein R¹⁶ and R¹⁷ are independently C₁-C₄alkyl, C₁-C₄haloalkyl or halogen.

5. A compound according to claim 1, wherein R¹ and R² are independently methyl or halomethyl.

6. A compound according to claim 1, wherein G¹ is O and G² is S.

7. A compound according to claim 1, wherein p is 1 and n is 1.

8. A compound of formula II 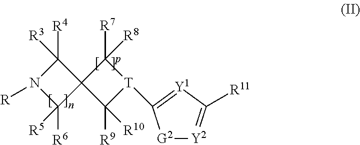
wherein R³, R⁴, R⁵, R⁶, R⁷, R⁸, R⁹, R¹⁰, R¹¹, G², T, Y¹, Y², n and p are as defined for a compound of formula I in claim 1, and R¹⁸ is hydrogen or a protecting group, or a salt or N-oxide thereof, or
a compound of formula III 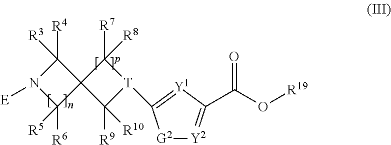
wherein E is hydrogen, a protecting group or group M 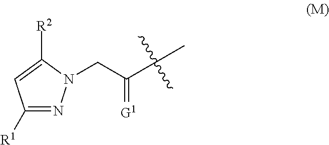
and R¹, R², R³, R⁴, R⁵, R⁶, R⁷, R⁸, R⁹, R¹⁰, G¹, G², T, Y¹, Y², n and p are as defined for a compound of formula I in claim 1, and R¹⁹ is C₁-C₆alkyl or a salt or N-oxide thereof, or
a compound of formula IV 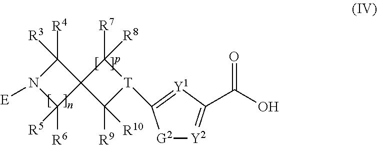
wherein E is hydrogen, a protecting group or group M 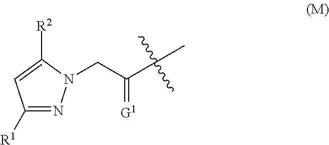
and R¹, R², R³, R⁴, R⁵, R⁶, R⁷, R⁸, R⁹, R¹⁰, G¹, G², T, Y¹, Y², n and p a are as defined for a compound of formula I in claim 1, or a salt or N-oxide thereof, or
a compound of formula V 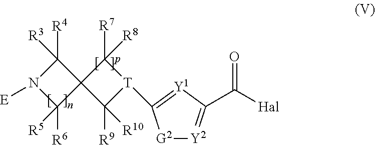
wherein E is hydrogen, a protecting group or group M, 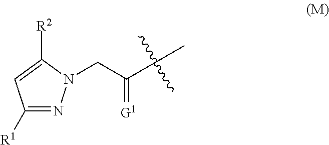
and R¹, R², R³, R⁴, R⁵, R⁶, R⁷, R⁸, R⁹, R¹⁰, G¹, G², T, Y¹, Y², n and p are as defined for a compound of formula I in claim 1, and Hal is halogen, preferably chloro or a salt or N-oxides thereof,
a compound of formula VI 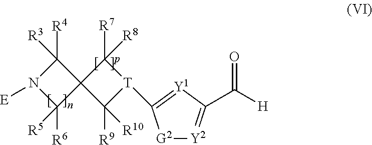
wherein E is hydrogen, a protecting group or group M 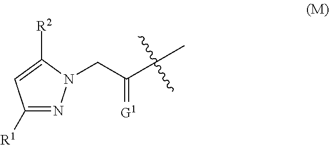
and R¹, R², R³, R⁴, R⁵, R⁶, R⁷, R⁸, R⁹, R¹⁰, G¹, G², T, Y¹, Y², n and p are as defined for a compound of formula I in claim 1, or a salt or N-oxides thereof.

9. A composition comprising at least one compound as defined in claim 1 and an agriculturally acceptable carrier, optionally comprising an adjuvant, and optionally comprising one or more additional pesticidally active compounds.

10. A method of controlling or preventing an infestation of plants, propagation material thereof, harvested crops or of non-living materials by phytopathogenic or spoilage microorganisms or organisms potentially harmful to man, which comprises the application of a compound as defined in claim 1, to the plant, to parts of the plants or to the locus thereof, to propagation material thereof or to any part of the non-living materials.
